(19) United States
(12) Patent Application Publication
(10) Pub. No.: US 2018/0086827 A1
(43) Pub. Date: Mar. 29, 2018

(54) THERAPEUTICS AND METHODS OF TREATING FIBROPROLIFERATIVE DISEASES

(71) Applicant: Board of Supervisors of Louisiana State University and Agricultural and Mechanical College, Baton Rouge, LA (US)

(72) Inventors: Anthony Wayne ORR, Benton, LA (US); Alexandra FINNEY, Bossier City, LA (US)

(21) Appl. No.: 15/706,636

(22) Filed: Sep. 15, 2017

Related U.S. Application Data
(60) Provisional application No. 62/558,807, filed on Sep. 14, 2017.
(60) Provisional application No. 62/394,872, filed on Sep. 15, 2016.

(51) Int. Cl. C07K 16/28; C12N 15/113; A61K 31/7105; A61K 39/395

(52) U.S. Cl. CPC C07K 16/28; C12N 15/1138; A61K 31/7105; A61K 2039/505; C07K 2317/76; C12N 2310/141; C12N 2320/31; A61K 39/39533

(57) ABSTRACT

A method of treating a condition in a mammal comprising: administering a pharmacologically effective amount of a therapeutic; wherein the therapeutic one of decreases EphA2 expression and inhibits ephrin type-A receptor 2; and the condition is a proinflammatory or fibroproliferative condition.

### CROSS REFERENCE TO RELATED APPLICATIONS/PRIORITY

The present invention claims priority to U.S. Provisional Patent Application Nos. 62/394,872 filed Sep. 15, 2016 and 62/558,807 filed Sep. 14, 2017, which are incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

The work described below was supported by Grant Nos. RO1 A156077 and P30GM110703 which were awarded by the National Institutes of Health. The Government has certain rights in the invention

### BACKGROUND

Atherosclerosis, a chronic inflammatory disease of the large arteries, involves the focal accumulation of low-density lipoproteins (LDL) in the vessel intima, stimulating endothelial cell activation and macrophage recruitment. As lipid accumulates, macrophage clearance of LDL becomes dysfunctional, resulting in foam cell formation visible histologically as fatty streaks. Smooth muscle fibroproliferative remodeling critically regulates the progression of atherosclerosis from early fatty streaks to advanced atheromas. As fatty streaks form, underlying medial smooth muscle cells undergo a phenotypic transition from their contractile, quiescent phenotype to a synthetic phenotype characterized by reduced contractile gene expression, enhanced cell proliferation and migration, and heightened deposition of extracellular matrix proteins. Although this fibroproliferative remodeling can be deleterious by enhancing plaque size to promote stenotic lesions, formation of a protective fibrous cap over the necrotic core of the plaque provides stability to the plaque by preventing rupture and thrombosis.

Atherosclerosis can cause coronary heart disease, angina, carotid artery disease; peripheral artery disease, and chronic kidney disease, for example. Although science has advanced in understanding of atherosclerosis, the condition is still implicated in the death of hundreds of thousands of individuals in the United States each year. For the foregoing reasons, there is a pressing, but seemingly irresolvable need for a method of treatment of atherosclerosis.

### SUMMARY

Wherefore, it is an object of the present invention to overcome the above mentioned shortcomings and drawbacks associated with the current technology.

The inventors present the first evidence that deletion of EphA2 in mouse models of atherosclerosis diminishes atherosclerotic plaque formation and limits atherosclerotic plaque progression.

The inventors demonstrated that EphA2 deletion reduces pro-inflammatory gene expression and early monocyte recruitment to the plaque, associated with reductions in monocyte firm adhesion under flow.

The inventors further demonstrated the first link between smooth muscle phenotypic modulation and EphA2 expression, and the inventors define a novel role for smooth muscle EphA2 expression in driving smooth muscle proliferation and extracellular matrix deposition.

Although EphA2 contributes to monocyte recruitment to the atherosclerotic plaque, EphA2 expression does not affect multiple other monocyte proinflammatory responses, suggesting that blunting EphA2 ligation may selectively reduce plaque-associated inflammation.

Although EphA2 expression also contributes to the smooth muscle fibroproliferative remodeling that drives the stable plaque phenotype, the effect of EphA2 on proliferation appears to be largely ligand independent, unlike inflammation. Blunting EphA2 ligation may limit inflammation while leaving smooth muscle fibroproliferative remodeling intact, thereby promoting a stable plaque phenotype.

The present invention relates to pharmaceutical compositions of a therapeutic (e.g., gene therapy, inhibitor), or a pharmaceutically acceptable salt, solvate, or prodrug thereof, and use of these compositions for the treatment of a proinflammatory or fibroproliferative condition, including atherosclerosis, acute lung injury, ischemia/reperfusion, cancer, and psoriasis.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a proinflammatory or fibroproliferative condition.

In certain embodiments, the proinflammatory or fibroproliferative condition is mild to moderate proinflammatory or fibroproliferative condition.

In further embodiments, the proinflammatory or fibroproliferative condition is moderate to severe proinflammatory or fibroproliferative condition.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the proinflammatory or fibroproliferative condition.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., therapeutic, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. N. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palm itate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a proinflammatory or fibroproliferative condition). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable such as those described in EP 1336602A1, which is herein incorporated by reference.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 shows EphA2 deletion reduces plaque size in multiple vascular beds. A through D, show plaque formation in mice fed a Western diet for 8 or 12 weeks was determined after Oil Red 0 staining. Representative micrographs are shown (A and C), and plaque area was calculated as a percent of the aorta staining positive for Oil Red 0 (B and D). n=7 to 9 male, 6 female. Scale bar=1 mm E and F, Male Apoe^{-/-} controls (EphA2 wild-type [WT]) and EphA2^{-/-}Apoe^{-/-} (EphA2 knockout [KO]) mice were fed a Western diet for 8 weeks, and the innominate and carotid arteries were stained with Movat pentachrome stain. Plaque area was quantified as the neointimal area within the internal elastic lamina. n=6 to 11 innominate artery, 8 to 11 carotid sinus. Scale bar=100 μm. Data are expressed as mean±SEM. Student t tests were used for statistical comparisons.

FIG. 2 shows reduced inflammation in early atherosclerotic plaque formation. Male EphA2 wild-type (WT) and EphA2 knockout (KO) mice were fed a Western diet for 8 weeks. A and B, Macrophage area (Mac2 positive, green) was determined by immunohistochemistry in both the innominate (n=6-10) and carotid (n=14-21) arteries. Smooth muscle (smooth muscle actin [SMA] positive, red) and endothelial (CD31 positive, white) cell stainings are also shown. Quantification of average macrophage area (B) is provided. Scale bar=100 μm. C and D, Relative gene expression between atherosclerosis-prone (aortic arch [AA]) and protected (thoracic aorta [TA]) regions was determined by mRNA isolation and quantitative real-time polymerase chain reaction and compared between EphA2 WT and EphA2 KO mice. Expression of (C) macrophage (CD68) and T-cell (CD3) marker genes or (D) markers of endothelial activation (vascular cell adhesion molecule-1 [VCAM-1], intercellular cell adhesion molecule-1 [ICAM-1], E-selectin) were normalized to the housekeeping genes PPIA and Rpl13a. n=5 to 8. Data are expressed as mean±SEM. Student t tests were used for statistical comparisons. eNOS indicates endothelial nitric oxide synthase.

FIG. 3 shows EphA2 does not contribute to macrophage inflammatory response or lipid uptake. A and B, EphA2 wild-type (WT) and EphA2 knockout (KO) mice were fed either standard chow (Chow) or Western diet (WD) for 8 weeks, and peritoneal macrophages were isolated after thioglycollate injection. A, mRNA isolated from WT and KO peritoneal macrophages was assessed for EphA and ephrinA family gene expression normalized to GAPDH. n=6. B, Quantification of peritoneal macrophages content. n=8 to 18 male, n=6 to 9 female. C through H, RAW264.7 macrophages were treated with Fc-ephrinA1 (1 μg/mL) for the indicated times, and changes in classic M1 markers (tumor necrosis factor-α [TNF-α], interleukin [IL]-1β, C-C chemokine receptor type 2 [CCR2]) and M2 markers (C-X-C chemokine receptor type 1 [CXCR1], IL-10, CD163) were assessed by quantitative real-time polymerase chain reaction (qRT-PCR). Results are normalized to Rpl13a and expressed as a fold change compared with unstimulated conditions. n=4. I, Peritoneal macrophages were treated with lipopolysaccharide (LPS; 100 ng/mL, 24 hours), and TNF-a gene expression was quantified by qRT-PCR and normalized to GAPDH. n=6. J, Uptake of Dil-oxidized low-density lipoprotein (Dil-oxLDL; 20 μg/mL, 4 hours) by WT and KO peritoneal macrophages was assessed. n=4. K and L, WT and KO mice were fed WD for 8 weeks, and the lipid levels in peritoneal macrophages was assessed by Nile Red staining, n=5 to 6. Scale bar=100 μm. Data are expressed as mean±SEM. Statistical comparisons were made with 2-way (B and I) or 1-way (C-H) ANOVA with Bonferroni posttest or Student t test (J and K).

FIG. 4 show EphA2 deletion from resident cells but not hematopoietic cells confers atheroprotection. A through E, EphA2 wild-type (WT) and EphA2 knockout (KO) mice were irradiated and received either EphA2 WT or KO bone marrow cells. Four weeks after irradiation, mice were fed a Western diet for 12 weeks. Atherosclerotic plaque formation was assessed in (A and B) the aorta by Oil Red O staining and (C-E) the innominate artery by immunohistochemistry for macrophages (Mac2; green), smooth muscle (smooth muscle actin [SMA]; red), and endothelial cells (CD31; white). Scale bar=1 mm (A) or 100 μm (C). n=5 to 7. D, Plaque area was quantified as the neointimal area within the internal elastic lamina. E, Macrophage area was quantified as the total area of positive Mac2 staining. F and G, Human aortic endothelial cells with or without EphA2 knockdown (siRNA) were treated with tumor necrosis factor-α (TNF-α; 10 ng/mL, 4 hours), and labeled primary human monocytes were perfused over the endothelial monolayer under physiological flow. Rolling velocity and firmly adherent monocytes were analyzed per high-powered field averaged over 6 fields. n=3 in duplicate. Data are expressed as mean±SEM. Statistical comparisons were performed with 1-way (B-E) or 2-way (F and G) ANOVA with Bonferroni posttests.

FIG. 5 shows EphA2 deletion reduces advanced atherosclerotic progression. A and B, EphA2 wild-type (WT) and EphA2 knockout (KO) mice were fed a Western diet for 16 weeks, and atherosclerosis in the aortas was visualized by Oil Red O staining. n=6 to 7. Scale bar=1 mm. C, Atherosclerotic plaques from innominate arteries and carotid sinuses were graded for plaque scores with a modified Stary scoring system. n=22 plaques from 8 WT mice, 18 plaques from 6 KO mice. D through F, Atherosclerotic plaque composition was assessed by immunohistochemistry for macrophages (Mac2; green) and smooth muscle cells (smooth muscle actin [SMA]; red). Total macrophage area and smooth muscle area were analyzed, and percent of the plaque area staining positive for SMA or Mac2 was calculated. Scale bar=100 μm. n=6 to 8. G and H, Human coronary artery smooth muscle cells (hCoASMCs) from 4 separate donors were either serum starved or treated with 10% complete media for 3 days. G, mRNA was isolated, and the expression of EphA and ephrinA family genes was quantified by quantitative real-time polymerase chain reaction and normalized to GAPDH. Representative expression of each gene was determined, and a heat map was generated. H, EphA2 protein expression was determined by Western blot and normalized to GAPDH. n=4. I, hCoASMCs were plated onto tissue culture dishes coated with collagen I (40 μg/mL), collagen IV (20 μg/mL), fibronectin (FN; 10 μg/mL), or laminin (LN; 20 μg/mL) and cultured for 3 days in serum-free conditions. EphA2 protein expression was assessed by Western blotting and normalized to GAPDH. n=5. J and K, Smooth muscle EphA2 expression in atherosclerotic plaques was assessed by immunohistochemistry. Plaques from (J) the carotid artery of Apoe^{-/-} mice fed a Western diet for 16 weeks or (K) grade V human atherosclerotic lesions from coronary arteries were stained for EphA2 (green) and smooth muscle cells (SMA; red). White arrows indicate medial smooth muscle cells. Scale bar=100 μm. n=4 to 5. Data are expressed as mean±SEM. Statistical comparisons were made with the Student t test (B, E, F, and H), 1-way ANOVA (I) with Bonferroni posttest, or X² test (C) comparing early-stage (I and II) and advanced-stage (III and IV) plaques.

FIG. 6 shows EphA2 deficiency attenuates proliferation in atherosclerotic lesions and human coronary artery vascular smooth muscle cells (hCoASMCs) in vitro. A through D, EphA2 wild-type (WT) and EphA2 knockout (KO) mice were fed a Western diet for 16 weeks, and proliferation in the (A and B) innominate and (C and D) carotid arteries was assessed by staining for Ki67 (red). The number of Ki67-positive cells in the plaque or adventitia was assessed, and plaque borders are indicated with a white dotted line. Scale bar=100 μm. n=6 to 7. E through H, hCoASMCs treated with or without EphA2 siRNA were maintained in either serum-free or 1% serum-containing conditions overnight, and proliferation was assessed by (E and G) staining for Ki67 (red) or (F and H) treatment with BrdU (10 μmol/L) for 2 hours and staining for BrdU (red). The percentage of proliferating cells was determined for each condition. n=4. Scale bar=100 μm. I and J, hCoASMCs were plated onto collagen I (40 μg/mL), and a scratch was created. Wound closure was monitored by time-lapse imaging (3 regions per wound) for 18 hours. Wound closure was calculated as percent closure from t=0. J, Representative images from scratch wound assay. Scale bar=100 μm. n=4. Data are expressed as mean±SEM. Statistical comparisons were made with the Student t test (B, D, G, and H) or 1-way repeated measures ANOVA (I) with Bonferroni post-test. *P<0.05, ** P<0.01, *** P<0.001, EphA2 siRNA vs mock control. HPF indicates high-powered field; and NT, not treated.

FIG. 7 shows EphA2 depletion decreases human coronary artery vascular smooth muscle cell (hCoASMC) mitogenic signaling in vitro and in atherosclerotic plaques. A through D, hCoASMCs were treated with or without EphA2 siRNA and maintained in 1% serum conditions overnight. A through C, For early time points (0-120 minutes), cells were serum starved 4 hours before 10% serum treatments at the indicated time points. Extracellular regulated kinase 1 and 2 (ERK1/2; A) or Akt1 (B) phosphorylation was determined by Western blot and normalized to total ERK1/2 or Akt1, respectively. Representative blots shown in C. After 24 hours of incubation in 1% serum, ERK1/2 (D) and Akt1 (E) phosphorylation was assessed by Western blot. F through I, EphA2 wild-type (WT) or EphA2 knockout (KO) mice were fed a Western diet for 16 weeks, and plaques from the innominate artery were assessed. F, Phospho-ERK1/2 (pERK1/2 positive; green) area or (H) phospho-Akt1 (pAkt1 positive; green) area was measured with smooth muscle cell area (smooth muscle actin [SMα-A] positive; red). Percent positive area of pERK1/2 (G) or pAkt1 (I) to total plaque or plaque-associated SMa-A was determined. Scale bars=100 μm. n=4 to 5. Data are expressed as mean±SEM. Statistical comparisons were made with 2-way ANOVA with Bonferroni posttest (A and B) or Student t test (D, E, G, and I). *Values for both mock and siEphA2 time points compared with their 0-minute controls. #Values only for mock time points compared with 0-minute controls. $Values for mock compared with siEphA2.

FIG. 8 shows deletion of EphA2 reduces plaque fibrosis in vivo and human coronary artery vascular smooth muscle cell (hCoASMC) matrix deposition in vitro. A through F, EphA2 wild-type (WT) or EphA2 knockout (KO) mice were fed a Western diet for 16 weeks, and the matrix remodeling in plaques from the innominate artery and carotid sinus was assessed by histology and immunohistochemistry. Collagen content was assessed with (A and B) Masson trichrome stain or (D and E) Picrosirius red stain, and the area of positive staining in the plaque was quantified. E and F, Fibronectin content was determined by immunohistochemistry for fibronectin (green), and fibronectin-positive area was quantified. n=6 to 8. Scale bar=100 μm. G and H, hCoASMCs treated with or without EphA2 siRNA were plated onto basement membrane proteins (Matrigel; 1:50 dilution) and maintained in either serum-free or 1% serum-containing conditions overnight. Cultures were fractionated into the deposited extracellular matrix (deoxycholate [DOC]-insoluble) fraction or the cell-associated (DOC-soluble) fraction. Total and extradomain A (EDA)-positive fibronectin (FN) was assessed in the DOC-soluble and DOC-insoluble fractions by Western blotting. n=4. Data are expressed as mean±SEM. Statistical comparisons were made with the Student t test (B, D, and F) or 1-way ANOVA (G and H) with Bonferroni posttests.

FIG. 9 is a table of quantitative real time PCR mouse primers.

FIG. 10 is a table of quantitative real time PCR human primers.

FIG. 11 shows EphA2 deletion reduces plaque size despite no alterations in early plaque composition. (A) Histogram showing percent of plaques in a given size range from vessels described in FIG. 1E and 1F. (B) Percent of plaques with specific Stary scores ranging from 0 to IV. n=32 plaques from 11 WT, n=19 plaques from 8 KO. C-G) EphA2 WT or EphA2 KO mice were fed Western diet for 8 weeks, and the aortic roots were assessed by immunohistochemistry. Movat pentachrome stain was used to assess plaque area (C), which was quantified as the neointimal area within the internal elastic lamina in (D). Plaque composition was assessed for macrophages (Mac2, green) and smooth muscle cells (SMA, red) (E). Total macrophage and smooth muscle area were analyzed (F), and percent of the plaque area staining positive for SMA or Mac2 was calculated (G). Scale bar=100 μm. n=4-5. Data are expressed as mean±SEM. Student's T-test was used for statistical comparison.

FIG. 12 shows enhanced weight gain and plasma lipid levels in EphA2-deficient mice. EphA2 WT and EphA2 KO mice were fed Western diet for up to 12 weeks. A/B) Blood glucose and body weight was measured weekly throughout Western diet feeding. C) After 8 weeks Western diet, total plasma cholesterol, triglyceride fractions, and HDL levels were determined. LDL was calculated according to Friedewald's equation. n=11-15 male, 8-11 female. D) Plasma was collected from male and female EphA2 WT and EphA2 KO mice fed a standard chow diet for up to 16 weeks, and enzymatic assays were used to detect total plasma cholesterol, triglyceride fractions, and HDL. LDL was calculated according to Friedewald's equation. n=13-17 males, 7-8 females. Data are expressed as mean±SEM. Two-way ANOVA with Bonferroni posttest was used for statistical comparison.

FIG. 13 shows EphA2 deletion does not affect macrophage phenotype. A) Mrna isolated from primary human peripheral blood monocytes (hPBM, n=4), THP-1 monocyte-like cell line (n=4), mouse peritoneal macrophages (mPerit. Mac, n=6), mouse bone marrow-derived macrophages (mBMDM, n=5), and the RAW264.7 macrophage cell line (n=3) was assessed for EphA/ephrinA family gene expression and normalized to GAPDH. Heat maps were produced to indicate the relative abundance of each gene product. B) Peritoneal macrophages were isolated from EphA2 WT and EphA2 KO mice following 8 weeks of standard chow or Western diet feeding. mRNA expression of classic M1 marker genes (CCR2, TNF-α) and M2 marker genes (CXCR1, IL-10, CD163, Fizz1) was assessed by qRT-PCR and normalized to GAPDH, n=6. CF) RAW264.7 macrophages were polarized toward an M1 phenotype (100 ng/mL LPS, 40 ng/mL IFN-γ) or M2 phenotype (40 ng/mL IL-4) for 0, 1, 3, 6, 12, and 24 hours. mRNA expression of EphA2 (C/E) was assessed by qRT-PCR and normalized to Rpl13a. Protein expression of EphA2 (D/F) was assessed by Western blotting and normalized to β-tubulin. n=3. Data are expressed as mean±SEM. One-way (C-F) and Two-way ANOVA (B) with Bonferroni post-test was used for statistical comparisons.

FIG. 14 shows macrophage EphA2 knockdown does not affect activation or lipid uptake. RAW264.7 macrophages were transfected with EphA2 siRNA using nucleofection, and A) EphA2 expression was determined with Western blot and normalized to GAPDH. n=4. B) 24 hours post-transfection, RAW264.7 macrophages were treated with oxLDL (25 ug/mL) for 24 hours and TNFα was measured by ELISA. C) Uptake of oxLDL was visualized with Nile Red. n=3. Data are means±SEM. Statistical comparisons were made using Two-way ANOVA with Bonferroni post-tests.

FIG. 15 shows hematopoietic EphA2 does not contribute to altered plasma lipid levels. Bone marrow chimeras were produced as previously described, and plasma was collected at the end of diet regimens. Plasma lipid levels were assessed using enzymatic assays. Data are means±SEM, n=6. Data are expressed as mean±SEM. One-way ANOVA with Bonferroni post-test was used.

FIG. 16 shows depletion of endothelial EphA2 attenuates THP-1 monocyte firm adhesion with no alterations in slow rolling. Human aortic endothelial cells with or without EphA2 siRNA were treated with TNFα (10 ng/mL, 4 hours) and labeled THP-1 monocytes were perfused over the endothelial monolayer under physiological flow velocity. Rolling velocity and firmly adherent monocytes were analyzed per field averaged over six fields. n=3 in duplicate. Data are expressed as mean±SEM. Two-way ANOVA with Bonferroni post-test was used for statistical comparisons.

FIG. 17 shows substrate-bound EphA2 enhances monocyte adhesion and spreading. A) Cell-Tracker Green-labelled THP-1 monocytes were plated onto glass coverslips coated with anti-human Fc with or without Fc-EphrinA1, Fc-EphA2, Fc-ICAM-1, and/or Fc-VCAM-1. Cells were allowed to adhere for 10 minutes. Cells were washed in HBSS and non-adhered cells collected. Monocyte adhesion was assessed by measuring fluorescence in the adherent and non-adherent pools and shown as percent monocyte adhesion. n=6. B/C) THP-1 monocytes were plated onto glass coverslips coated with anti-human Fc with or without fibronectin, Fc-EphrinA1, or Fc-EphA2. Cells were allowed to adhere and spread for 1 hour, after which cells were fixed. Cells were stained for active β1 integrin (9EG7, red) and phalloidin (green), and spread cell area was determined using phalloidin staining and quantified with NIS Elements software. n=3. Scale bars=50 μm. D) Atherosclerotic plaques in ApoE−/− mice were stained for phosphorylated EphA2 (Phospho-Y772, green), macrophages (Mac2, red), and cell nuclei (DAPI, blue). Data are expressed as means±SEM, One-way ANOVA with Bonferroni post-test was used.

FIG. 18 shows EphA2 Deletion Reduces Atherosclerotic Plaque

Area and Smooth Muscle Content. A-E) EphA2 WT or EphA2 KO mice were fed Western diet for 8 weeks, and the aortic roots were assessed by immunohistochemistry. Movat pentachrome stain was used to assess plaque area (A), which was quantified as the neointimal area within the internal elastic lamina in (B). Plaque composition was assessed for macrophages (Mac2, green) and smooth muscle cells (SMA, red) (C). Total Mac2 and SMA positive area (D) and the percent of plaque area staining positive for Mac2 and SMA (E) were analyzed. N=5-8. F) EphA2 WT or EphA2 KO mice were fed Western diet for 16 weeks, and the innominate arteries were assessed by immunohistochemistry. Smooth muscle content was measured by immunohistochemistry for smooth muscle actin (SMa-A, red) and smooth muscle myosin heavy chain (SM-MHC, green). Total smooth muscle area (G) was analyzed, and percent of the plaque area staining positive for SMA or SM-MHC (H) was calculated. n=6-8. Scale bar=100 μm. Data are expressed as mean±SEM. Student's T-test was used for statistical comparisons.

FIG. 19 shows EphA2 deletion does not alter plaque-associated angiogenesis. AD) EphA2 WT and EphA2 KO mice were fed Western diet for 24 weeks to induce large atherosclerotic plaques prone to angiogenesis. A/C) Blood vessels in the right carotid artery and aortic root were labeled by biotinylated tomato lectin (LEA, green) injection 5 minutes prior to euthanasia and by immunohistochemistry for the endothelial marker Von Willebrand factor (vWF, red). B/D) Positive intraplaque or adventitial staining for lectin or vWF-positive vessels was assessed and expressed as a percent of the total plaque area or total adventitial area. Scale bar is 50 μm in (A), 100 μm in (B). n=5-7. Data are means±SEM, Student's T-test was used.

FIG. 20 shows EphA2 expression corresponds to the induction of the smooth muscle synthetic phenotype. hCoASMCs were cultured in either serum-free or 10% complete media-containing conditions for 3 days. A) Smooth muscle myosin heavy chain (SM-MHC), B) smooth muscle actin (SMA), and C) calponin were assessed by Western blot and normalized to GAPDH as an indicator of phenotypic modulation, with representative blots in (D). mRNA expression of SMA, MHC11, and Calponin was assessed by qRT-PCR and normalized to GAPDH (E). n=4. F, G) hCoASMCs were cultured in serum-free media for 3 days to induce quiescence and then treated with (F) 10% complete media the indicated time points or (G) increasing serum concentrations for 24 hours. n=3-4. Data are expressed as mean±SEM. Student's T-test was used in (A-E), and One-way ANOVA with Bonferroni post-tests were used for statistical comparisons in (F/G).

FIG. 21 shows alterations in EphA2 expression with synthetic phenotype in human aortic smooth muscle cells. hAoSMCs were cultured in either serum-free or 10% complete media-containing conditions for 3 days. A) Smooth muscle myosin heavy chain (SMMHC), B) smooth muscle actin (SMA), and C) calponin were assessed by Western blot and normalized to GAPDH as an indicator of phenotypic modulation, with representative blots in (D). mRNA expression of SMA, MHC11, and Calponin was assessed by qRT-PCR and normalized to GAPDH (E). F) EphA2 protein expression was determined by Western blot and normalized to GAPDH. I) hAoSMCs were plated onto collagen I (40 μg/mL), collagen IV (20 μg/mL), fibronectin (10 μg/mL), or laminin (20 μg/mL) and cultured for 3 days in serum-free conditions. EphA2 protein expression was assessed by Western blotting and normalized to GAPDH. n=3-4. Data are expressed as mean±SEM. Student's T-test was used in (A-F), and One-way ANOVA with Bonferroni post-test was used for statistical comparisons in (G).

FIG. 22 shows EphA2 knockdown reduces proliferation similarly with a different EphA2 siRNA and in human aortic smooth muscle cells. A/B) hCoASMCs were treated with or without Sigma EphA2 siRNA. C/D) hAoSMCs were treated with or without Dharmacon EphA2 siRNA. Cells were maintained in either serum-free or 1% serum-containing conditions overnight, and proliferation was assessed by staining for Ki67 (red) (A,C), and the percentage of proliferating cells was determined (B,D). n=3. Scale bar=100 μm. Data are expressed as mean±SEM. One-way ANOVA with Bonferroni post-test was used for statistical comparisons.

FIG. 23 shows validation of EphA2 knockdown with two siRNAs.

hCoASMCs were treated with (A/B) Sigma EphA2 siRNA or (C/D) Dharmacon EphA2 siRNA. 24 hours posttransfection, mRNA expression of EphA2 was assessed by qRT-PCR and normalized to GAPDH (A,C). n=3. EphA2 and SMA protein expression were determined by Western blot and normalized to GAPDH. n=4. Data are expressed as mean±SEM. Student's T-test was used for statistical comparisons.

FIG. 24 shows EphA2 deficiency reduces mitogenic signaling in hAoSMCs and in carotid plaques. A/B) hAoSMCs were treated with or without EphA2 siRNA and maintained in 1% serum conditions. 24 hours' post-transfection, cells were assessed for ERK1/2 and Akt1 phosphorylation by Western blotting. (A) ERK1/2 or (B) Akt1 phosphorylation was calculated as fold change from mock and normalized to total ERK1/2 or Akt1, respectively. n=4. Students Ttest was used for statistical comparison. C-F) EphA2 WT or EphA2 KO mice were fed Western diet for 16 weeks, and plaques from the carotid artery were assessed by histology and immunohistochemistry. C) Phospho-ERK1/2 area (pERK1/2 positive, green) or (E) phospho-Akt1 area (pAkt1 positive, green) was determined by immunohistochemistry in the carotid arteries (n=4). Smooth muscle (SMA-positive, red) cell staining are also shown. Percent positive area to total plaque or plaque SMA of phospho-ERK1/2 (D) or phospho-Akt1 (F) is provided. Scale bar=100 μm. Data are expressed as mean±SEM. Student's T-test was used for statistical comparisons.

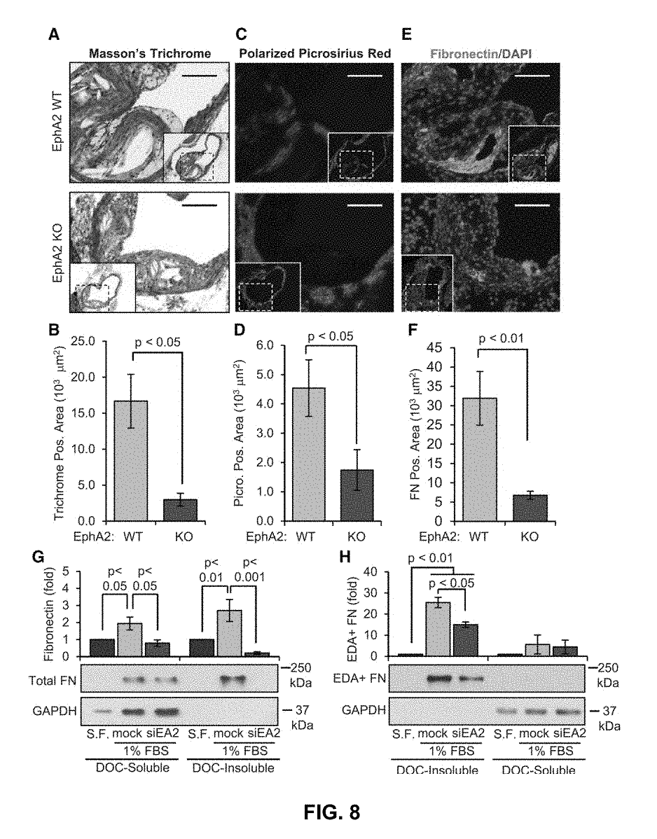

FIG. 25 shows EphA2 knockout reduces fibrosis. EphA2 WT or EphA2 KO mice were fed Western diet for 16 weeks, and matrix remodeling in plaques from the innominate and carotid arteries were assessed. Collagen content was measured as described previously, and the area of positive trichrome (A) or Picrosirius red (B) staining in the plaque was quantified as percent of plaque area. Fibronectin content was determined by immunohistochemistry for fibronectin (green), and fibronectin-positive area was quantified as percent of plaque area (C). Representative images are FIGS. 8A/C/E. n=6-8. Data are expressed as mean±SEM. Student's T-tests were used for statistical comparisons.

FIG. 26 shows EphA2 knockout reduces fibrosis in the aortic root. A/D) Apoe−/− mice (WT) or Apoe−/− , EphA2-/mice (KO) were fed Western diet for 16 weeks, and matrix remodeling in plaques from the aortic root were assessed by histology. Collagen content was assessed using Masson's Trichrome stain (A) or Picrosirius Red (D), and the area of positive staining in the plaque was quantified as total area (B/E) or percent of plaque area (C/F). n=5-8. Scale bar=100 μm. Data are expressed as mean±SEM. Student's T-tests were used for statistical comparisons.

FIG. 27 shows EphA2 knockdown blunts fibronectin deposition but not expression. A) hCoASMCs were transfected with or without EphA2 siRNA. The following day, cells were switched to either serum-free or 1% FBS for 24 hours, and mRNA expression of collagen I (Col1a1, Col1a2), collagen III (Col3a1), collagen VIII (Col8a1), and fibronectin (FN) was assessed by qRT-PCR and normalized to GAPDH. n=4. B/C) hCoASMCs treated as in (A) were removed using deoxycholate (DOC) buffer and the DOC-insoluble matrix was assessed by immunocytochemistry for total fibronectin (B, blue) or EDA+ fibronectin (C, blue). D, E) Fibronectin-positive area was measured using NIS Elements 3.0 software, and represented as fold change from serum-free conditions. n=4. Scale bar is 20 μm. Data are means±SEM. Oneway ANOVA with Bonferroni post-tests were used for statistical comparisons.

FIG. 28 shows reduced fibronectin deposition with an alternative EphA2 siRNA and in hAoSMCs. hCoASMCs (A) or hAoSMCs (B/C) were treated with or without Sigma EphA2 siRNA (A) or Dharmacon EphA2 siRNA (B/C) and plated onto basement membrane proteins (Matrigel, 1:50 dilution) and maintained in either serum-free or 1% serum-containing conditions overnight. Cultures were fractionated into the deposited extracellular matrix (deoxycholate (DOC)-insoluble) fraction or the cell-associated (DOC-soluble) fraction as previously described. Total or extradomain A (EDA)-positive fibronectin were assessed in the DOC-soluble and DOC-insoluble fractions by Western blotting. n=4. Data are expressed as mean±SEM. One-way ANOVA with Bonferroni post-tests were used for statistical comparisons.

FIG. 29 shows EphA2 knockdown does not alter MMP activity. hCoASMCs were treated with or without EphA2 siRNA. 24 hours post-transfection, cells were plated onto collagen I (40 μg/mL) or fibronectin (10 μg/mL) and allowed to incubate overnight. mRNA expression of MMP2 and MMP9 were assessed by qRT-PCR and normalized to GAPDH (A/B). n=3-5. MMP activity was assessed by gelatin zymography. MMP2 activity (˜72 kDa band on zymogram gel) was calculated as fold change from mock on collagen I and normalized to total MMP2 expression. Since no bands were observed at approximately 100 kDa on the zymogram gel, total MMP9 (92 kDa) protein expression was not shown. n=3. Data are means±SEM. Students T-test (A/B) or Two-way ANOVA with Bonferroni post-tests (C) were used for statistical comparisons.

FIG. 30 shows EphA2 knockdown does not affect wound closure when on fibronectin. hCoASMCs were maintained in either serum-free or 1% conditions and treated with or without EphA2 siRNA. 24 hours post-transfection, cells were plated onto fibronectin (10 μg/m L) and a scratch wound was induced. Wound closure was measured by time-lapse videomicroscopy (3 areas per wound, 30 minute intervals) for 18 hours and calculated as percent wound closure from t=0. n=4. Data are means±SEM. Two-way repeated measures ANOVA with Bonferroni post-tests were used for statistical comparisons.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

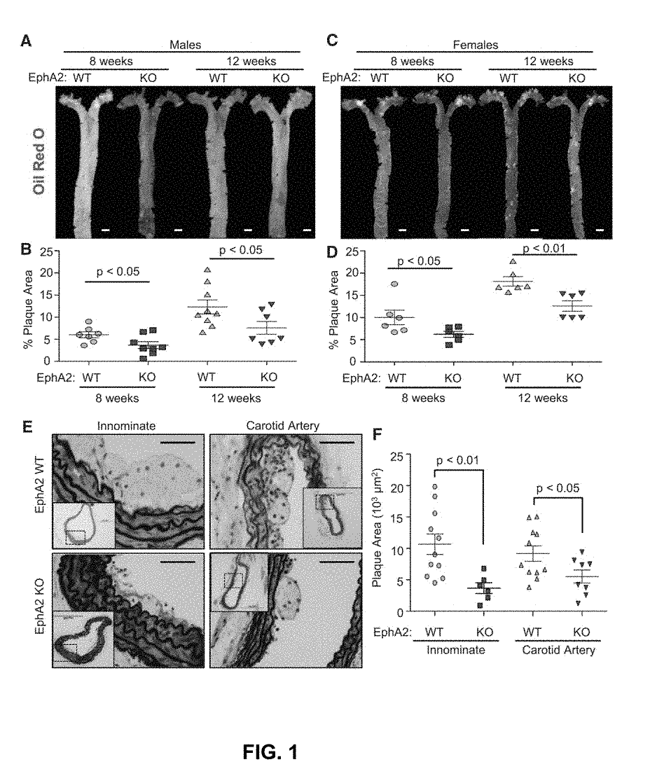

Turning now to FIGS. 1A-30, a brief description concerning the various components of the present invention will now be briefly discussed.

Plaque Analysis and Immunohistochemistry: The aortic root, aorta, innominate artery, and carotid arteries were harvested for analysis of atherosclerotic plaque burden. All paraffin-embedded tissues were sectioned at 5-μm thickness. Stains were visualized on a Nikon Eclipse Ti inverted epifluorescence microscope equipped with a photometrics CoolSNAP120 ES2 camera and NIS Elements 3.00, SP5 imaging software. Analysis was performed with NIS Elements software. Scale bars are provided in figure legends.

Cell Culture and Treatments: Human peripheral blood monocytes were isolated with Percoll and Ficoll density gradients from blood drawn from human donors following a LSU Health Sciences Center Institutional Review Board approved protocol. Peritoneal macrophages were isolated after thioglycollate injection. Human aortic endothelial cells (Lonza) and RAW264.7 macrophages (ATCC) were cultured as previously described. LDL (Intracel) was oxidized as described previously.

Lipid Uptake: Peritoneal macrophages isolated from mice fed a high-fat Western diet for 8 weeks were stained with Nile Red to assess lipid content. Uptake of Dil-oxidized LDL was also assessed with peritoneal macrophages and quantified with fluorescence microscopy.

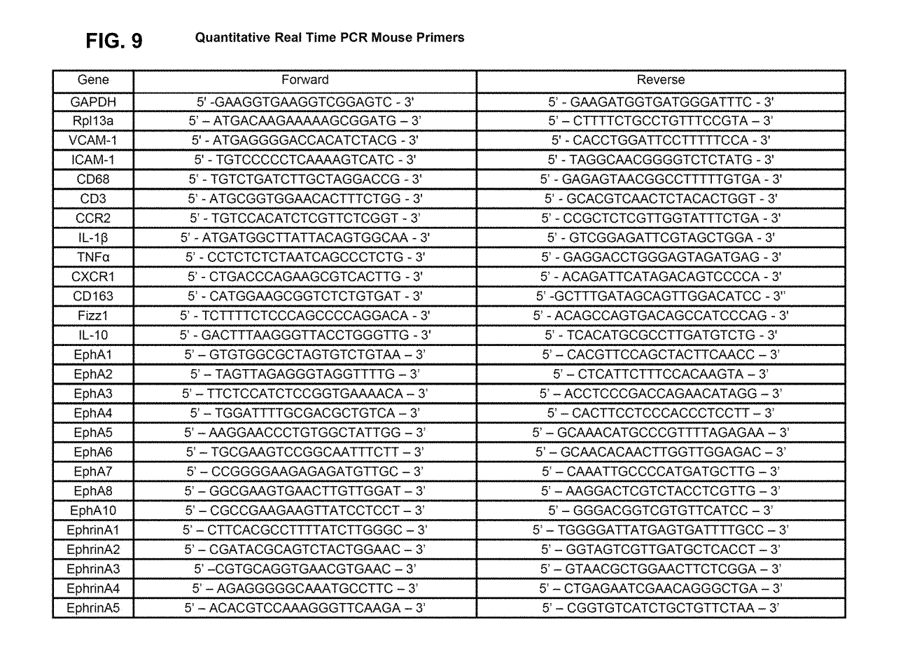

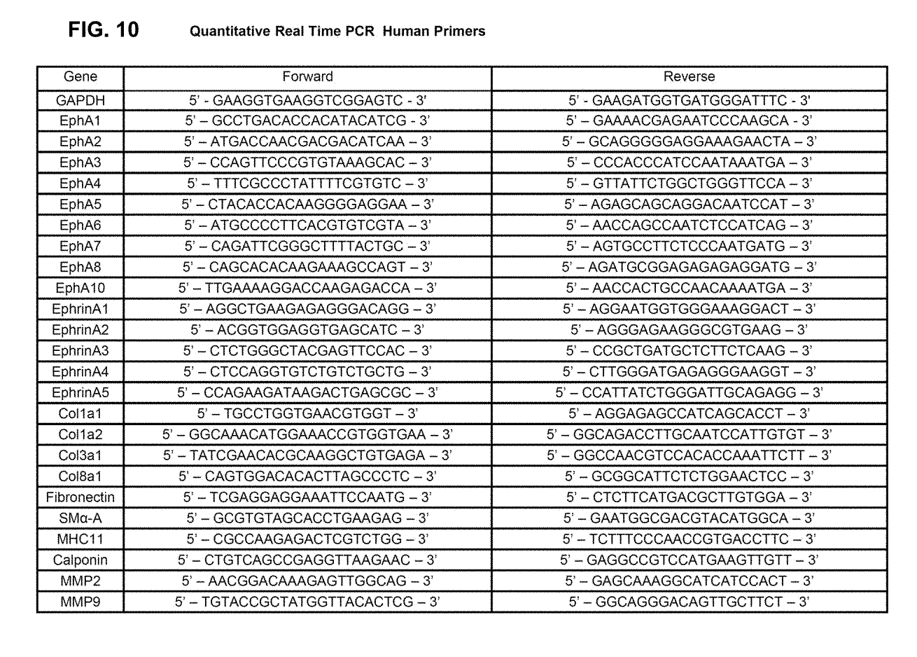

Quantitative Real-Time Polymerase Chain Reaction: Quantitative real-time polymerase chain reaction was performed as described previously. mRNA was extracted with TRIzol reagent, and cDNA was synthesized with the iScript cDNA synthesis kit (BioRad). Quantitative real-time polymerase chain reaction primer sequences can be found in FIGS. 9 and 10.

Immunoblotting and Immunocytochemistry: Cell lysis, Western blotting, and tumor necrosis factor-α (TNF-α) ELISA assays (eBioscience) were performed as described previously.

Matrix Deposition Analysis: Deoxycholate extraction was performed as described previously.

Statistical Analyses: All statistical analyses were performed with

GraphPad Prism Software. The number of experiments performed and statistical test used are provided in the figure legends. Values are expressed as mean±SEM.

RESULTS: EphA2 Deletion Reduces Plaque Size in Multiple

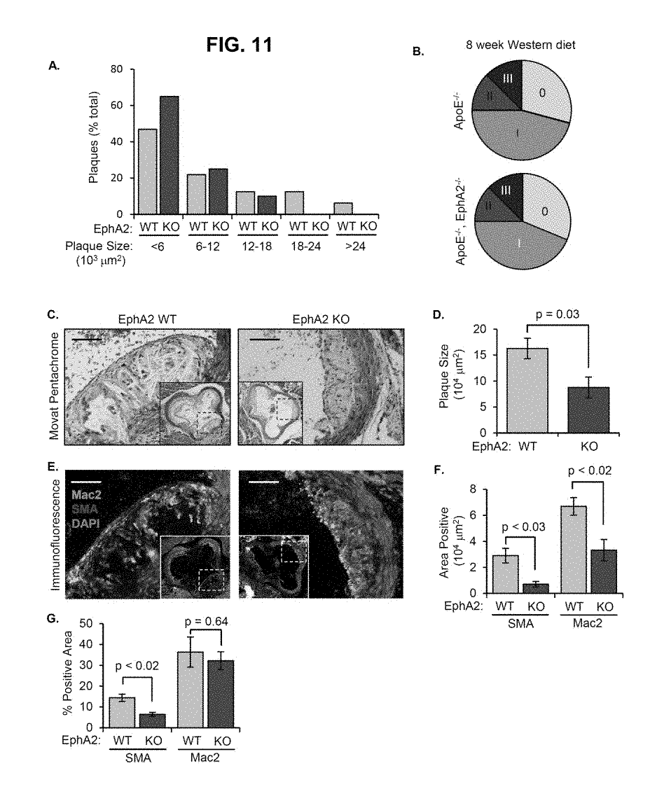

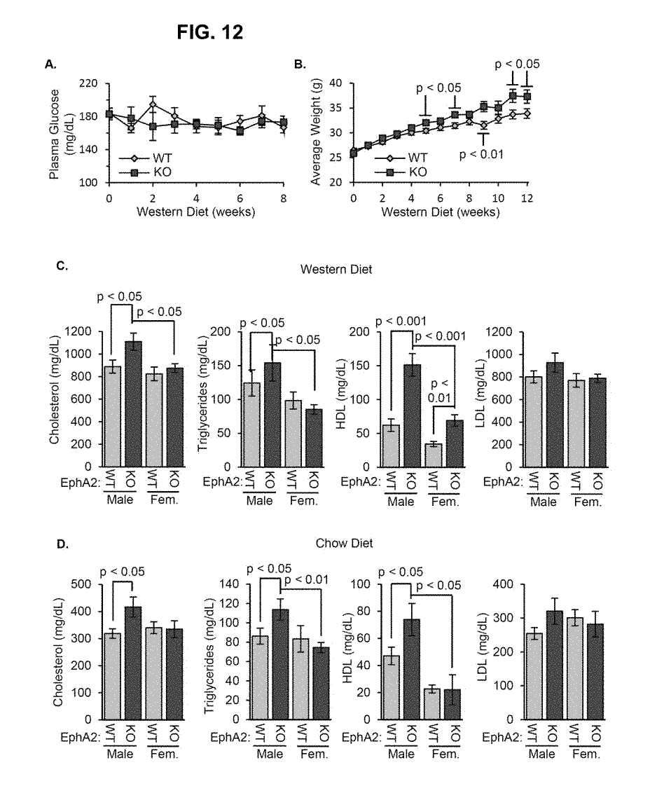

Vascular Beds: To determine whether EphA2 contributes to atherosclerosis, the inventors crossed EphA2^{-/-} mice with Apoe^{-/-} mice prone to hypercholesterolemia and atherosclerosis. Apoe^{-/-} (EphA2 wild type [WT]) and EphA2^{-/-}Apoe^{-/-} (EphA2 knockout [KO]) mice were fed a high-fat Western diet for up to 12 weeks. Mouse weight and blood glucose were monitored weekly, and plasma lipid profile and atherosclerotic plaque formation were assessed at the 8- and 12-week time points. For the initial analysis, both male and female mice were used to assess sex-specific differences in plaque formation after EphA2 deletion. However, both male and female EphA2 KO mice showed reduced atherosclerotic plaque formation at the 8- and 12-week time points as assessed by Oil Red O staining of the mouse aorta (FIGS. 1A-1D), so subsequent analysis focused on plaque characterization in male mice. Although effects on plaque formation may differ by vascular site, plaque area in cross sections of the innominate artery and carotid sinus were similarly reduced in male EphA2 KO mice, suggesting that EphA2 deletion reduces atherosclerosis across multiple vascular beds (FIGS. 1E and 1F). Although EphA2 KO mice showed a predilection for smaller plaques (FIG. 11A), the stage of plaque progression (classic Stary scoring system) remained similar between EphA2 KO and EphA2 WT mice after 8 weeks of a Western diet (FIG. 11B). Blood glucose levels remained similar between groups (FIG. 12A), but EphA2 KO mice demonstrated enhanced weight gain (FIG. 12B) compared with EphA2 WT controls. Furthermore, despite reductions in atherosclerotic plaque formation, analysis of plasma lipids levels showed elevated total cholesterol, high-density lipoprotein cholesterol, and plasma triglycerides in male EphA2 KO mice compared with EphA2 WT mice (FIG. 12C), whereas female mice demonstrated a similar lipid profile between the 2 groups. Similarly exacerbated plasma lipid profiles were observed in male EphA2 KO mice, but not female EphA2 KO mice, fed a standard chow diet (FIG. 12D). The similar reduction in plaque size between male and female EphA2 KO mice suggests that the slight elevation in plasma high-density lipoprotein observed in male EphA2 KO mice likely does not contribute to the atheroprotective effect.

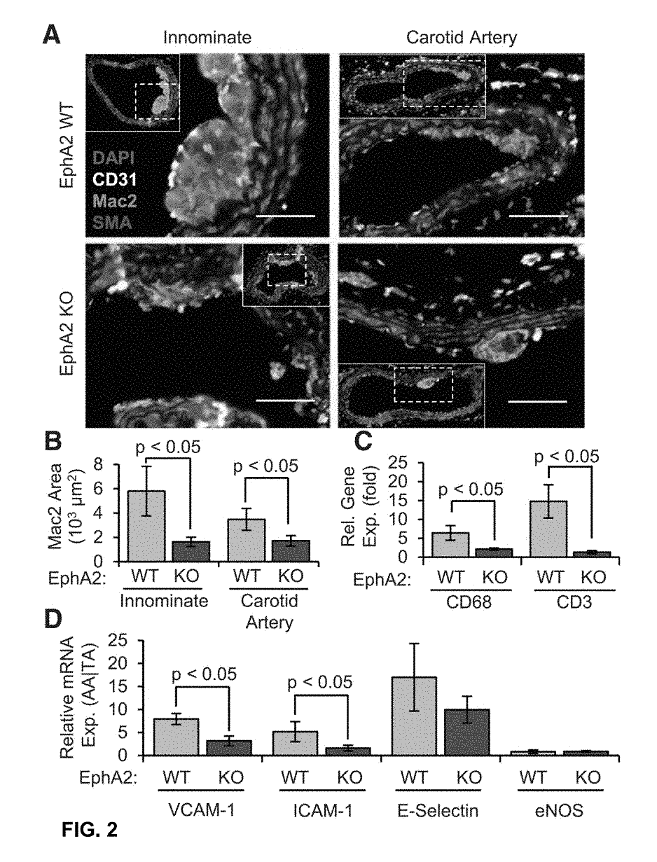

EphA2 Deletion Reduced Macrophage Accumulation and Inflammatory Gene Expression In Vivo: The inventors are aware that EphA2 is implicated in inflammation in a variety of systems. The inventors compared leukocyte accumulation and proinflammatory gene expression during early atherogenesis between EphA2 WT and EphA2 KO mice. After 8 weeks of a Western diet, macrophage area (Mac2 positive) was reduced in atherosclerotic lesions from EphA2 KO mice in both the innominate and carotid arteries compared with EphA2 WT mice (FIGS. 2A and 2B). Similar reductions in plaque area and macrophage area were observed in the aortic root of EphA2 KO mice, after EphA2 siRNA injection (FIGS. 11C-11G). mRNA isolated from the atherosclerosis-prone aortic arch of EphA2 WT mice showed elevated CD68 expression (macrophage marker) and CD3 expression (T cells) compared with the atherosclerosis-resistant thoracic aorta (FIG. 2C); however, this induction was significantly reduced in EphA2 KO mice, indicative of reduced leukocyte content. In addition, markers of endothelial activation (ICAM-1, VCAM-1) showed a similar reduction in EphA2 KO mice compared to EphA2 WT mice (FIG. 2D). Together, these data suggest that EphA2 deletion reduces endothelial activation and leukocyte accumulation during early atherosclerosis.

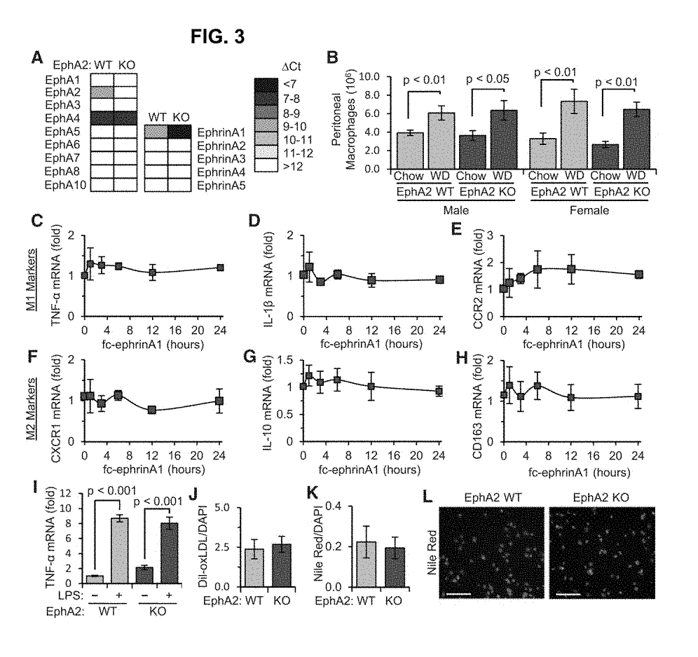

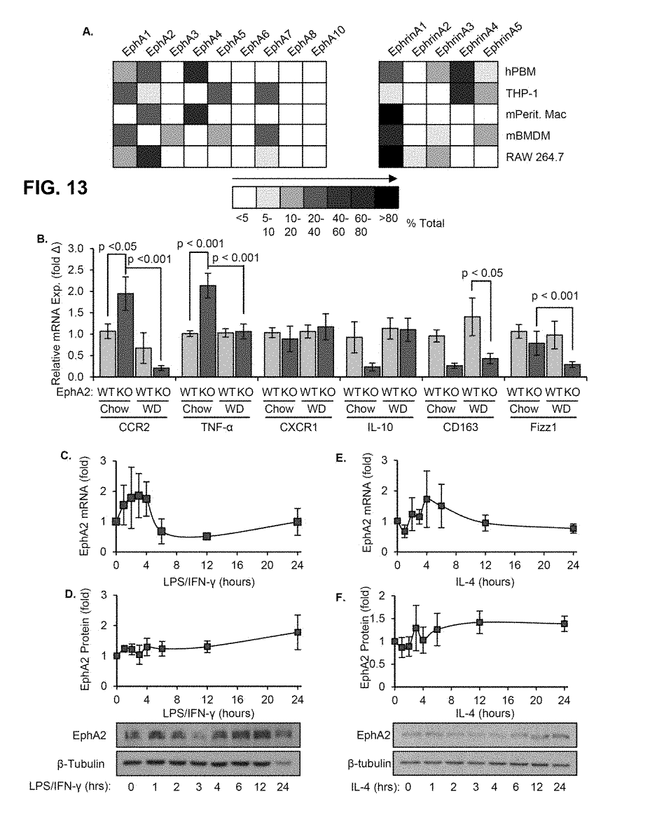

Deletion of EphA2 Has No Effect on Macrophage Gene Expression or Lipid Uptake In Vitro: Because plaque macrophages express EphA2, the inventors next sought to determine whether EphA2 contributes to macrophage function and gene expression. The inventors assessed the Eph/ephrin gene expression profile and observed similar expression profiles in human peripheral blood monocytes, murine peritoneal macrophages, and the RAW264.7 human macrophage cell line, with prominent monocyte/macrophage expression of EphA2, EphA4, and ephrinA1 (FIG. 13A). However, bone marrow-derived macrophages and the THP-1 monocyte-like cell line showed a disparate Eph/ephrin expression profile, limiting their usefulness for assessing EphA2 function in macrophages (FIG. 13A). Thioglycollate-elicited peritoneal macrophages isolated from EphA2 KO mice showed ablated EphA2 expression without compensatory upregulation of EphA4 (FIG. 3A). In contrast, ephrinA1 expression was enhanced in EphA2 KO mice, consistent with antagonistic expression patterns between EphA2 and ephrinA1.

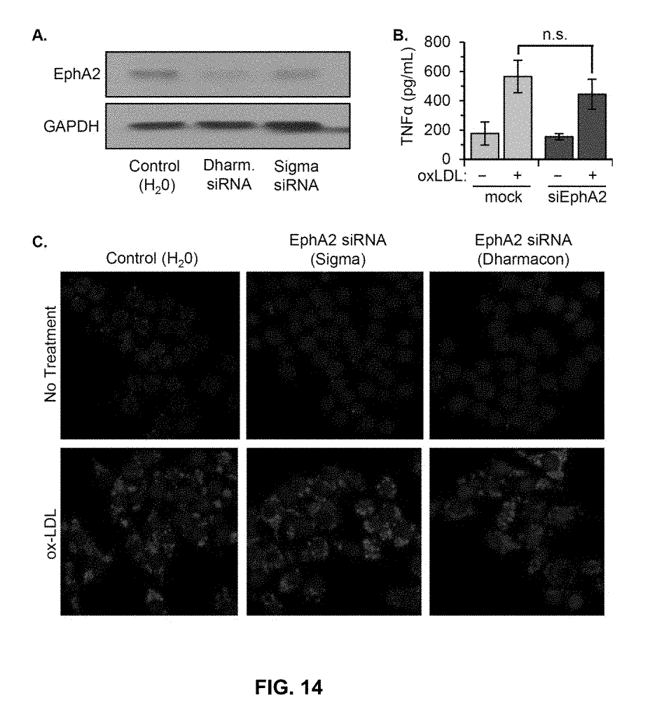

Because EphA2 deletion has shown both positive and negative effects on leukocyte homing in multiple model systems, the inventors analyzed macrophage infiltration after thioglycollate-induced sterile inflammation in the peritoneal cavity. Although Western diet-fed mice exhibited enhanced peritoneal macrophage recruitment in both models, there was no difference in macrophage recruitment between EphA2 KO mice and EphA2 WT mice regardless of diet (FIG. 3B). Analysis of M1 and M2 marker gene expression showed no consistent trends between EphA2 KO and EphA2 WT peritoneal macrophages (FIG. 13B), and ligation of EphA2 in RAW264.7 macrophages with soluble ephrinA1 did not affect M1 or M2 marker expression (FIGS. 3C-3H). Furthermore, polarization of RAW264.7 macrophages toward M1 or M2 phenotypes did not affect EphA2 expression (FIGS. 13C-13F). Although peritoneal macrophages from EphA2 KO mice showed enhanced mRNA expression of the M1 marker gene TNF-α under chow-fed conditions (FIG. 13B), this association was lost after Western diet feeding. Furthermore, EphA2 KO peritoneal macrophages showed no difference in lipopolysaccharide-induced TNF-α expression (FIG. 31), and siRNA-mediated EphA2 knockdown in RAW264.7 macrophages did not affect TNF-a induction by oxidized LDL (FIGS. 14A and 14B). Similarly, EphA2 KO peritoneal macrophages and EphA2-depleted RAW264.7 macrophages showed no deficiency in Dil-oxidized LDL uptake (FIGS. 3J and 14C), and peritoneal macrophages from EphA2 KO mice and EphA2 WT mice showed no difference in lipid uptake, identified by Nile Red staining, after Western diet feeding (FIGS. 3K and 3L). Taken together, these data suggest that EphA2 signaling does not significantly affect macrophage phenotype, proinflammatory gene expression, or lipid uptake.

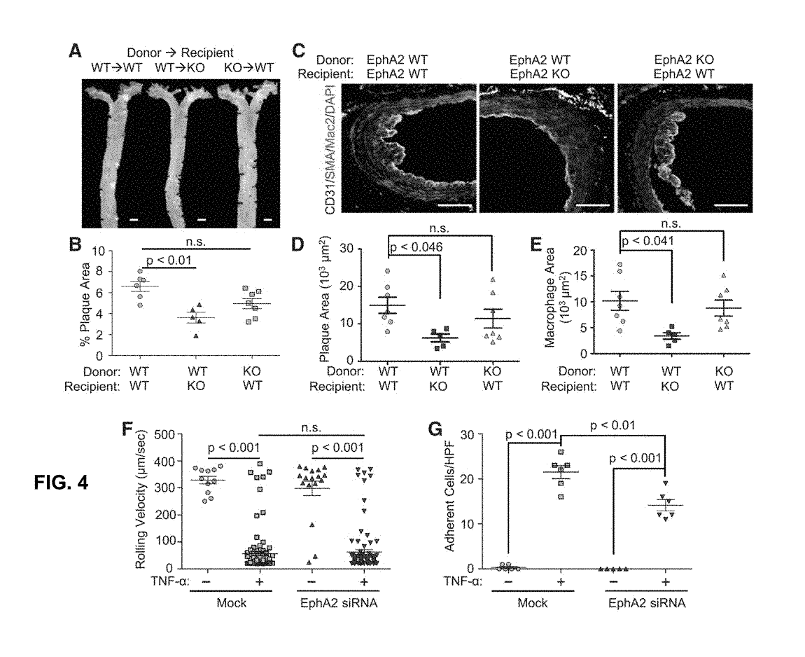

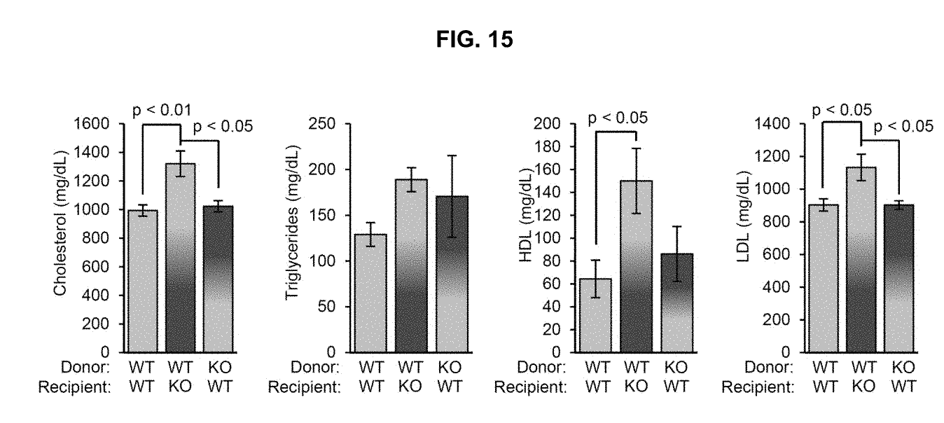

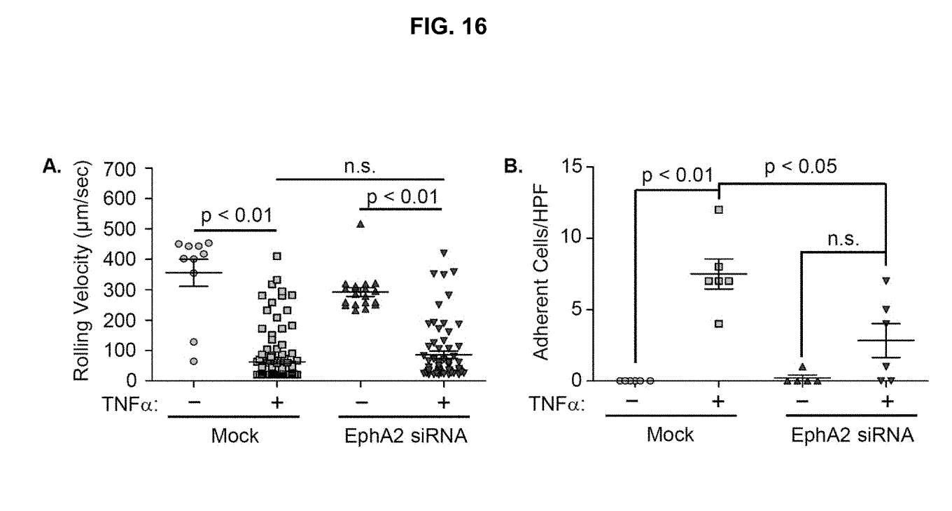

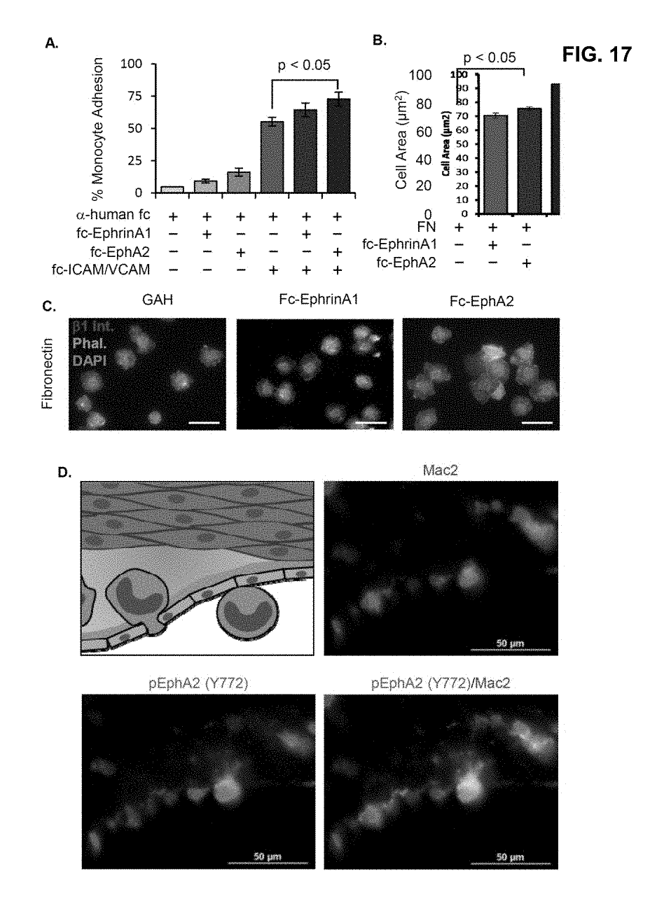

Deletion of EphA2 Confers Protection Against Atherosclerosis in Resident Lineages Compared With Hematopoietic Lineage: To determine the specific contributions of EphA2 in hematopoietic or resident cell lineages in vivo, bone marrow chimeras were generated and atherosclerotic plaque formation was assessed after 12 weeks on a Western diet. Alterations in hematopoietic EphA2 expression did not affect plasma lipid levels in this model, suggesting that the disparate lipid profile likely derives from changes in hepatic lipid handling and not altered reverse cholesterol transport (FIG. 15). Consistent with our in vitro data suggesting EphA2 does not significantly affect macrophage function, EphA2 KO mice still showed reduced plaque formation when reconstituted with EphA2 WT bone marrow (FIGS. 4A and 4B), whereas EphA2 WT mice reconstituted with EphA2 KO bone marrow showed only minor, insignificant reductions compared with reconstitution with EphA2 WT marrow. Cross-sectional analysis of atherosclerotic plaques in the innominate artery showed significant reductions in plaque size only when EphA2 was deleted in resident lineages and not with EphA2-deleted bone marrow (FIGS. 4C and 4D). Similarly, plaque macrophage area was significantly reduced in EphA2 KO mice even when reconstituted with EphA2 WT bone marrow (FIGS. 4C and 4E), whereas plaque macrophage content was unaltered in EphA2 WT mice whether reconstituted with EphA2 WT or EphA2 KO marrow. Although these data suggest that vascular EphA2 expression plays a primary role in regulating atherosclerotic inflammation, we cannot rule out a role for EphA2 expression in macrophages because no statistically significant differences were observed between EphA2 deletion in the recipient mice and EphA2 deletion in the marrow-derived cells. Supporting a primary role for endothelial EphA2 in atherogenic inflammation, EphA2 knockdown in human aortic endothelial cells reduced TNF-α-induced firm adhesion of human peripheral blood monocytes (FIGS. 4F and 4G) and THP-1 monocytes (FIGS. 16A and 16B) under flow conditions but had no effect on monocyte rolling. Indeed, monocytes exhibit enhanced spreading and adhesion when plated onto recombinant EphA2 in vitro but showed no effect when plated onto recombinant ephrinA1 ligand (FIGS. 17A-17C). EphA2/ephrinA1 interactions induce EphA2 phosphorylation, and prominent EphA2 phosphorylation (Y772) was observed in plaque endothelial cells bound to monocytes (FIG. 17D). However, EphA2 does not appear to regulate monocyte targeting to all vascular beds because monocyte recruitment to the peritoneal cavity after thioglycollate injection was similar between EphA2 WT and EphA2 KO mice (FIG. 3B). Taken together, these data suggest that endothelial EphA2 contributes to monocyte recruitment into atherosclerotic lesions.

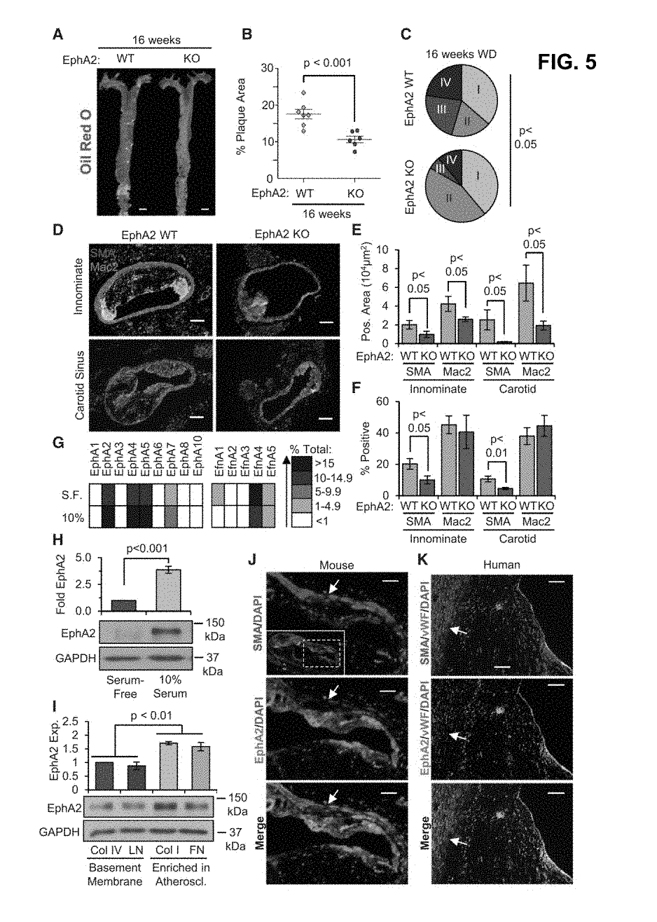

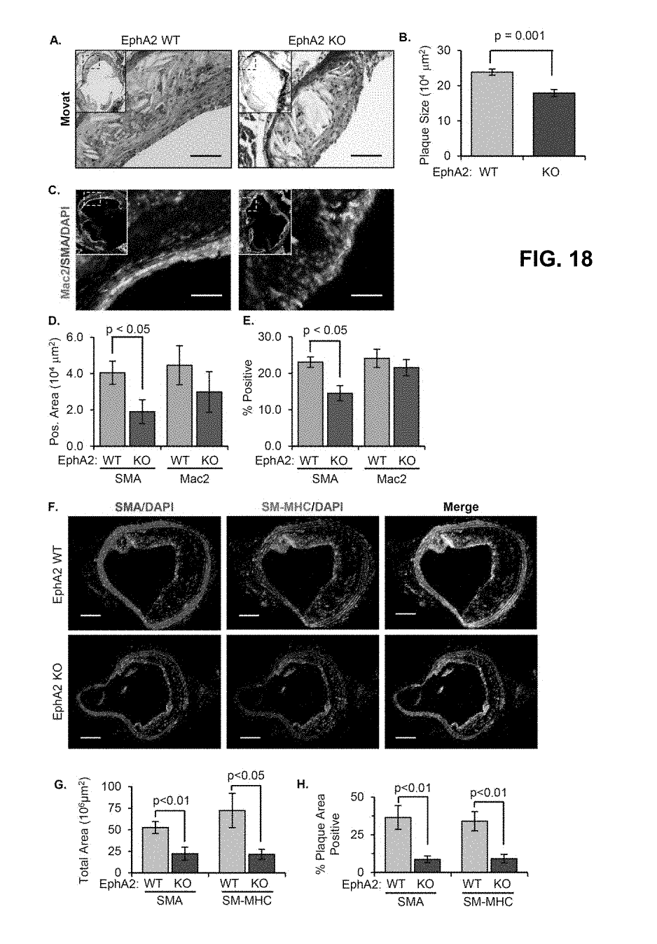

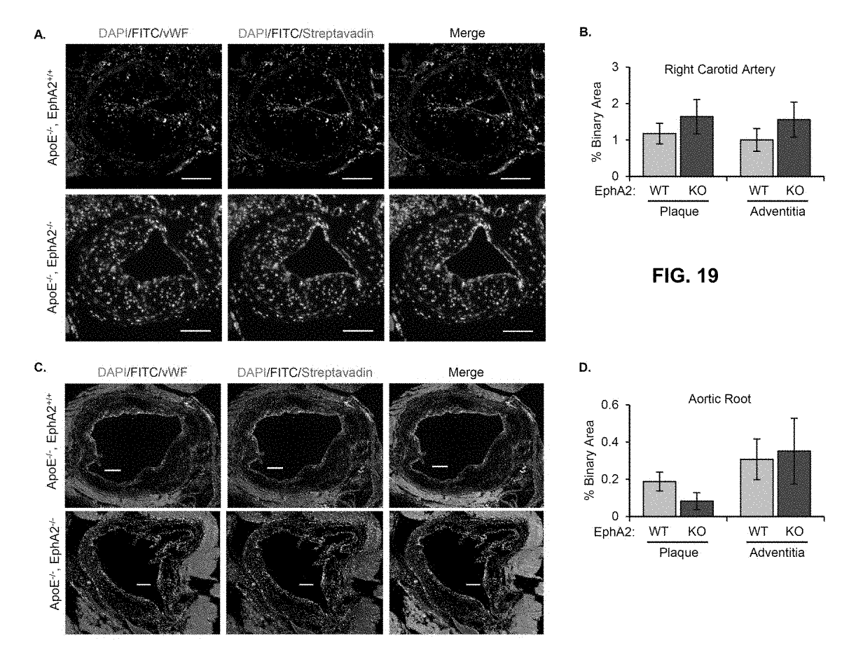

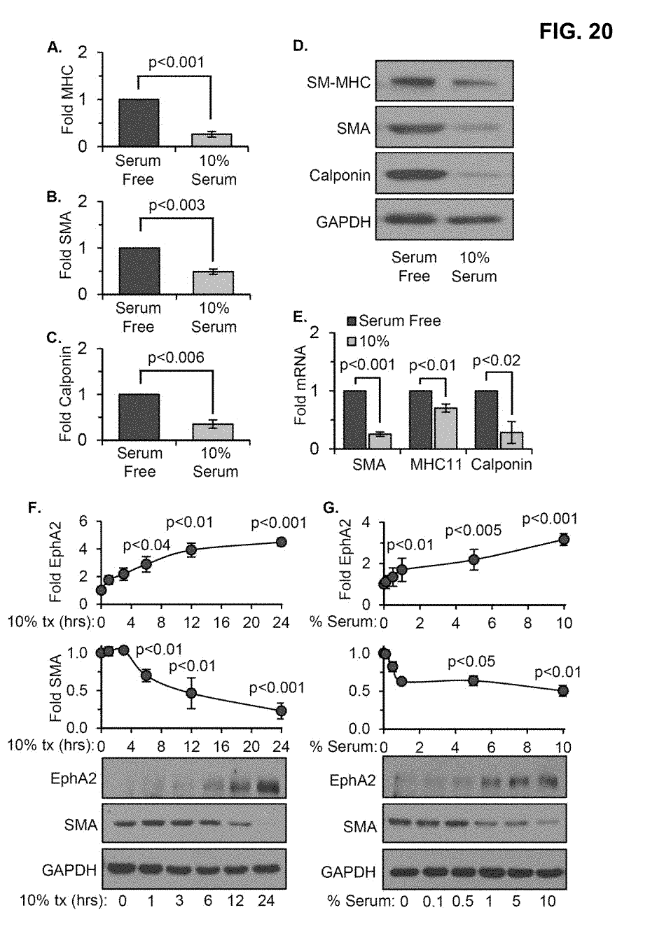

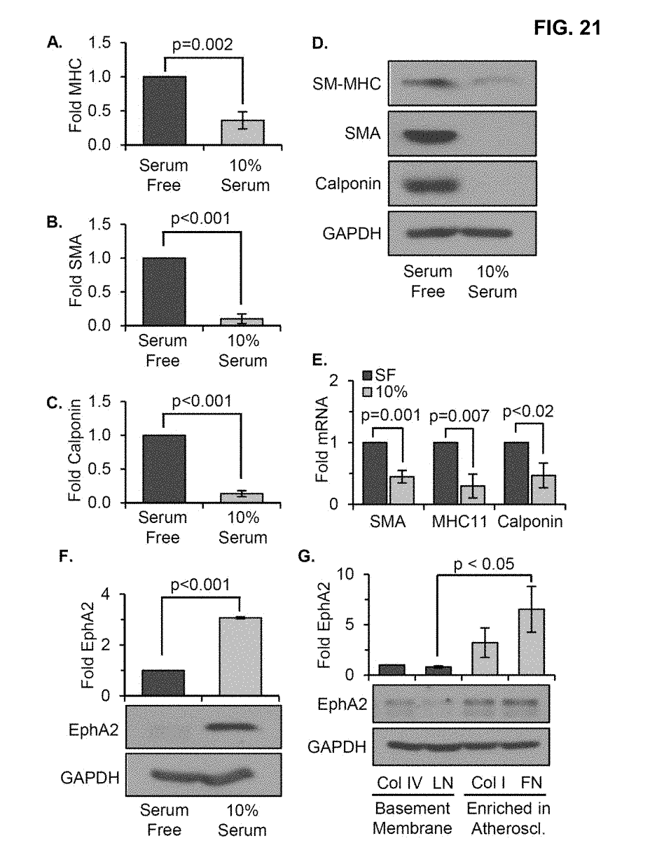

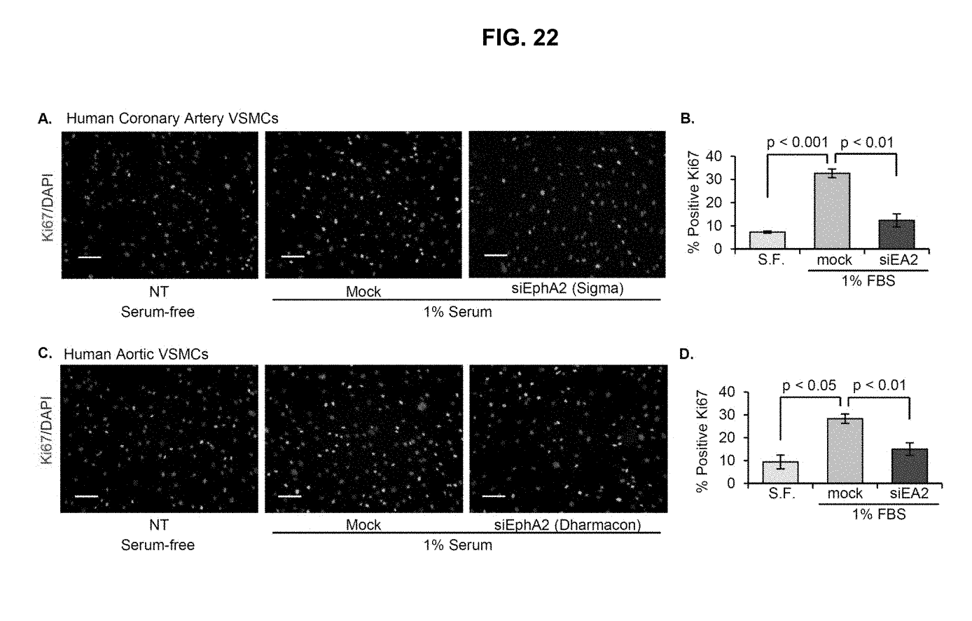

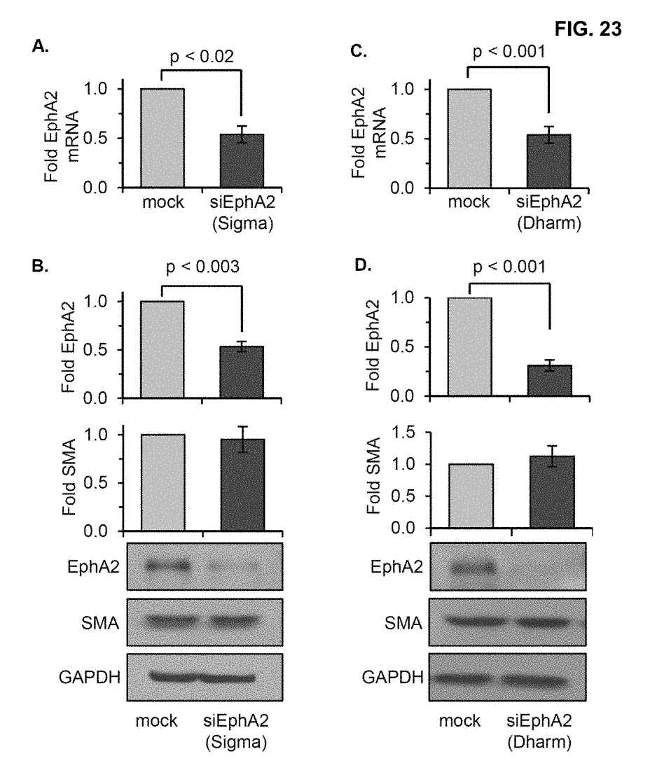

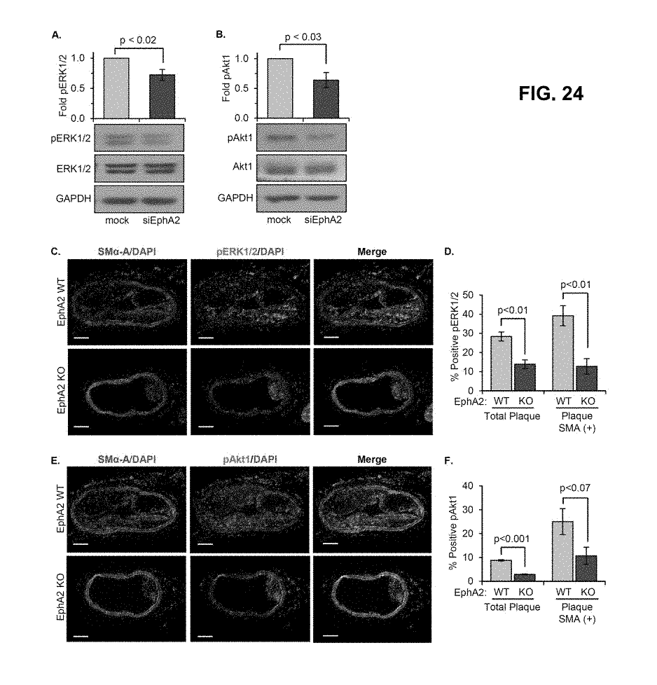

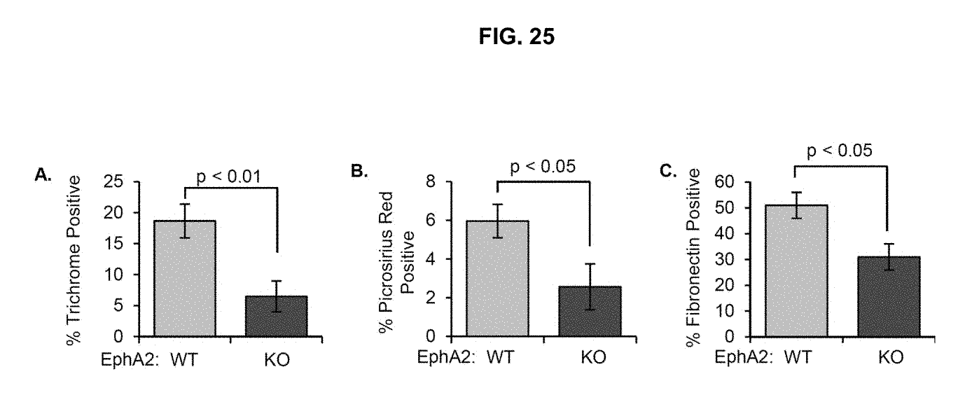

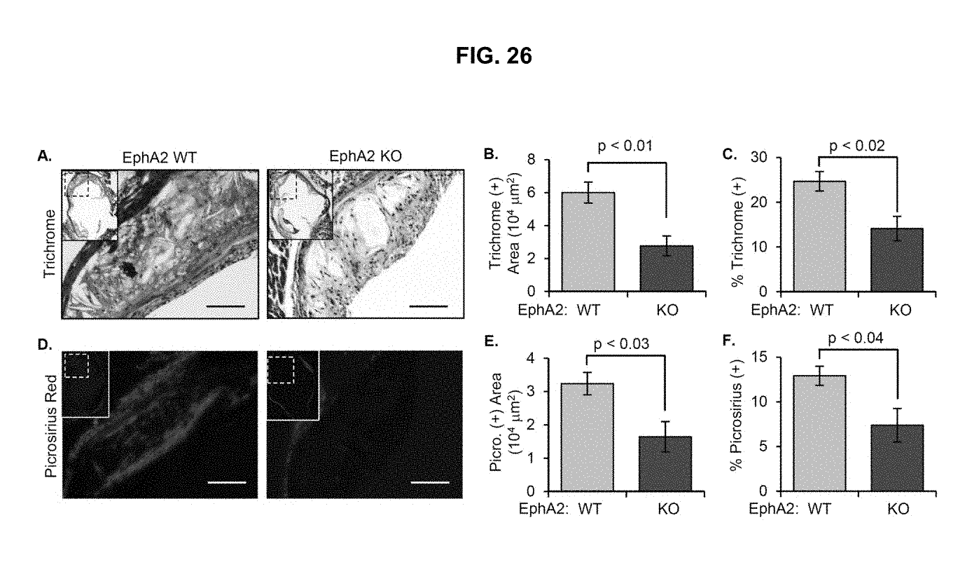

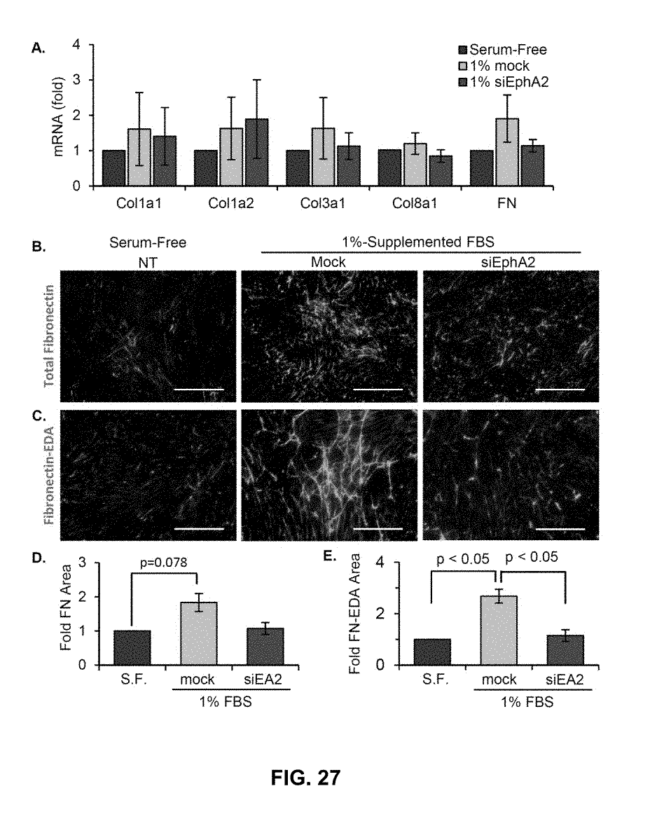

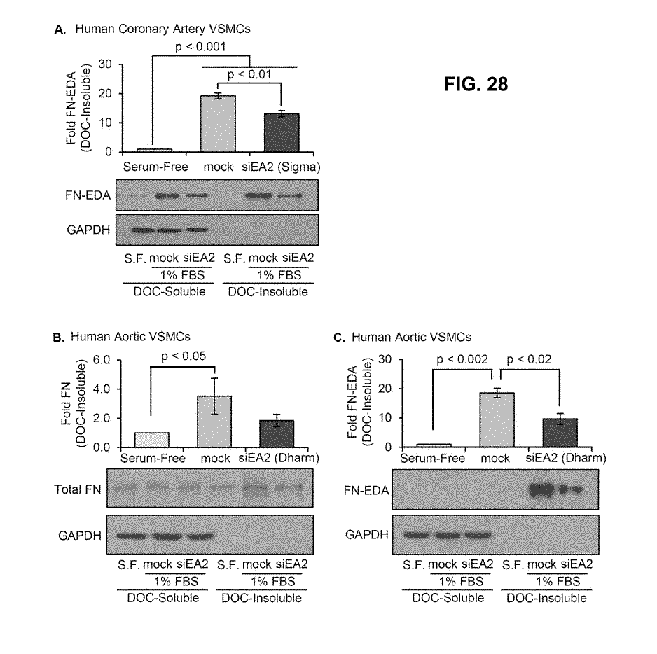

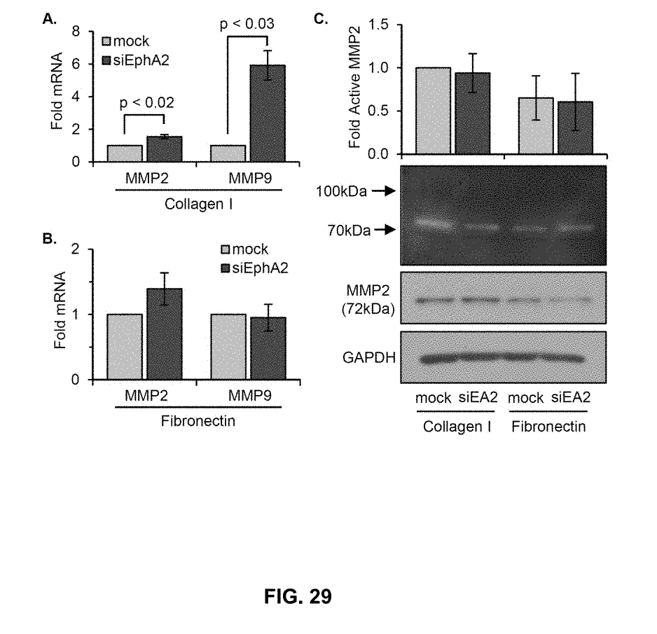

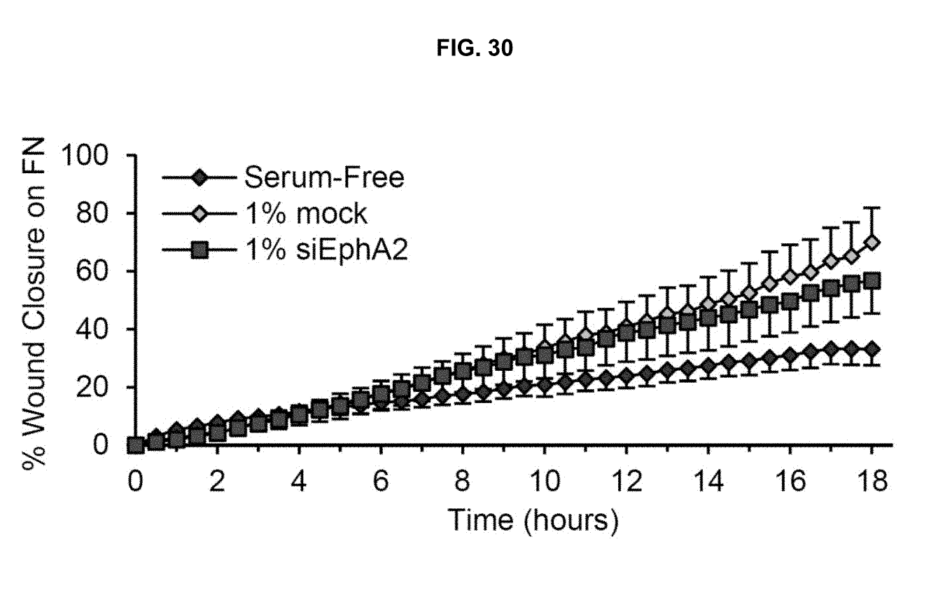

EphA2 Deletion Attenuates Progression to Advanced Atherosclerotic Plaques: Although EphA2 deletion did not affect the stage of plaque progression at early time points after Western diet feeding (8 weeks), we next investigated whether EphA2 contributes to plaque progression to advanced atherosclerosis after 16 weeks of Western diet feeding. Consistent with earlier time points, deletion of EphA2 reduced plaque area as demonstrated by Oil Red O staining of the aorta (FIGS. 5A and 5B). Analysis of plaque stage with the Stary scoring system revealed a reduced incidence of advanced atheromas (type III and IV plaques) in EphA2 KO mice compared with EphA2 WT controls (FIG. 5C). The reduction in plaque progression was associated with attenuated smooth muscle actin-positive smooth muscle area in the atherosclerotic lesions from the innominate artery, carotid artery (FIGS. 5D-5F), and aortic root (FIGS. 18A-18D). Consistent with this staining pattern, EphA2 KO mice also show reduced staining for the smooth muscle marker gene smooth muscle myosin heavy chain in the atherosclerotic plaques (FIGS. 18E and 18F), suggesting that EphA2 expression affects smooth muscle incorporation into the atherosclerotic lesion. Although plaque angiogenesis is associated with advanced atherosclerotic plaques and endothelial EphA2 contributes to angiogenesis, the inventors did not detect any differences in capillary density in either the plaque or vessel adventitia in EphA2 KO mice compared with EphA2 WT mice (FIG. 11A-19D).

EphA2 Shows Differential Expression During Vascular Smooth Muscle Phenotypic Modulation: Because vascular smooth muscle phenotypic modulation critically regulates atherosclerotic plaque progression, the inventors next sought to define whether EphA2 expression is altered during smooth muscle phenotypic modulation in vitro and in vivo. In cell culture, vascular smooth muscle cells maintain the contractile, quiescent phenotype in serum-depleted conditions but transition to a synthetic phenotype in response to serum (FIGS. 20A-20D). Human coronary artery vascular smooth muscle cells (hCoASMCs) expressed several EphA receptors and ephrinA ligands, including EphA2, EphA4, EphA5, EphA7, ephrinA1, ephrinA4, and ephrinA5. Serum-induced transition to the synthetic phenotype promoted the mRNA expression of EphA receptors while reducing the expression of ephrinA ligands (FIG. 5G) and promoted EphA2 protein expression in a dose-dependent and time-dependent manner (FIGS. 5H, 20F, and 20G). The inventors observed a similar phenotypic transition in human aortic smooth muscle cells (hAoSMCs) (FIGS. 21A-21E) associated with enhanced EphA2 expression (FIG. 21F). In addition to serum, smooth muscle cells undergo phenotypic transition in response to a variety of extracellular matrix proteins, with basement membrane proteins (collagen IV, laminin) promoting the quiescent, contractile phenotype and plaque-associated interstitial matrix proteins (unpolymerized collagen I, fibronectin) promoting a synthetic phenotype. Like the smooth muscle response to serum, culturing either hCoASMCs or hAoSMCs on collagen I and fibronectin promoted EphA2 expression compared with cells on basement membrane proteins (FIGS. 5I and 21G). Consistent with in vitro models, plaque-associated smooth muscle cells (smooth muscle actin positive) showed enhanced EphA2 expression compared with medial smooth muscle cells in both murine carotid and human coronary atherosclerotic lesions (FIGS. 5J and 5K). Together, these data suggest that smooth muscle cells in the atherosclerotic plaque express EphA2.

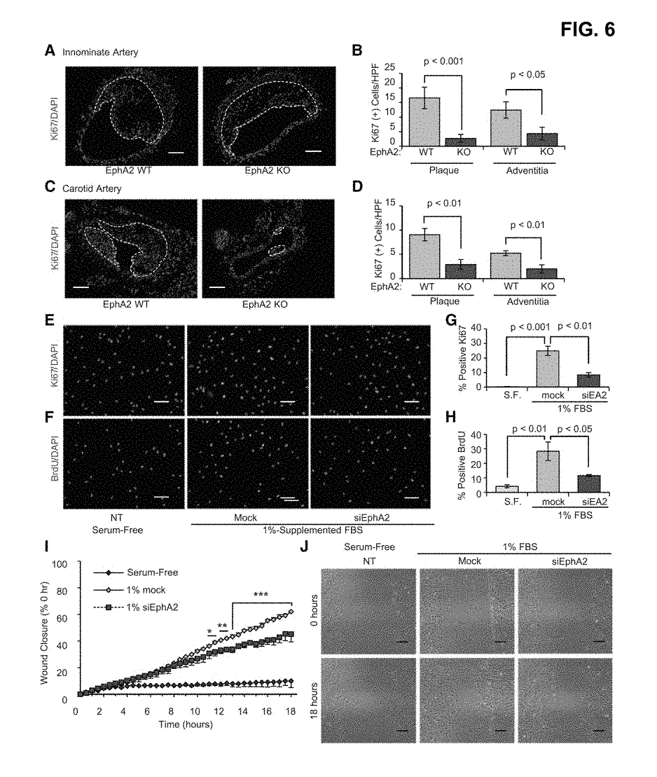

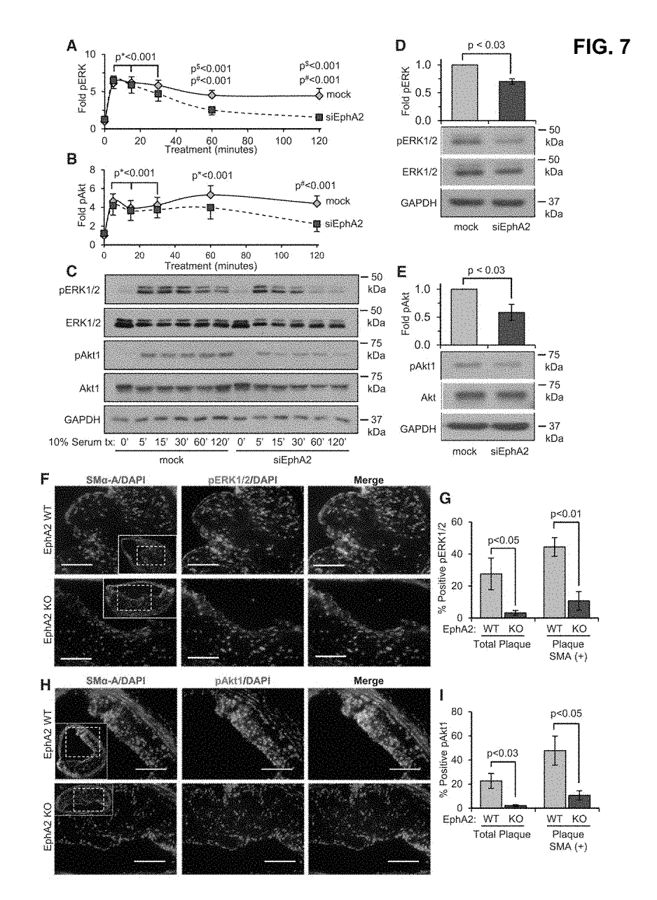

EphA2 Depletion Reduces Proliferation and Matrix Remodeling in Atherosclerotic Plaques and Vascular Smooth Muscle Cells: During atherosclerotic progression, migration and proliferation of smooth muscle cells into the neointima contribute to plaque size. Several known genetic loci for cardiovascular disease involve genes that regulate cell proliferation and carcinogenesis, and EphA2 upregulation contributes to proliferation in a variety of cancer models. Therefore, the inventors sought to determine whether EphA2 expression affects proliferation within atherosclerotic lesions. Using Ki67 as a marker for proliferation, immunohistochemistry showed a significant reduction in proliferation within the atherosclerotic lesions of EphA2 KO mice compared with EphA2 WT mice in both the innominate (FIGS. 6A and 6B) and carotid (FIGS. 6C and 6D) arteries, and this reduction was apparent in both the plaque itself and vessel adventitia. Consistent with reduced proliferation in the absence of EphA2, siRNA-mediated EphA2 knockdown in hCoASMCs reduced both Ki67 staining (FIGS. 6C and 6D) and BrdU incorporation (FIGS. 6E and 6F) in response to serum. Similar reductions in smooth muscle Ki67 staining were observed in hAoSMCs and with a second siRNA construct (FIGS. 22A-22D), and EphA2 knockdown was verified at both at the mRNA and protein level for both siRNAs (FIGS. 23A-23D). In addition, time-lapse videomicroscopy of hCoASMC wound healing in the presence of 1% serum shows a significant reduction in scratch wound closure in EphA2 siRNA-treated cells that was apparent at 11 hours after scratch and maintained for at least 18 hours (FIGS. 6G and 6H), suggesting that maximal serum-induced proliferation and scratch wound closure require EphA2 expression.

The inventors are aware of a variety of cancer models where enhanced

EphA2 expression promotes activation of extracellular regulated kinase (ERK1/2) and Akt1, classic mitogenic signaling pathways, through ligand-independent receptor signaling. To assess whether EphA2 expression affects mitogenic signaling in smooth muscle cells, the inventors assessed serum-induced signaling responses after EphA2 knockdown with siRNA. hCoASMCs were serum deprived for 24 hours after EphA2 knockdown, followed by stimulation with serum either short term (120 minutes) or long term (24 hours). Although EphA2 knockdown did not affect the early induction of either ERK1/2 or Akt1 phosphorylation in response to serum (5-30 minutes), EphA2 depletion greatly diminished the sustained activation of these pathways at the later time points (FIGS. 7A-7C). Similarly, lower levels of ERK1/2 and Akt1 phosphorylation were observed after 24 hours of serum stimulation in both hCoASMCs (FIGS. 7D and 7E) and hAoSMCs (FIGS. 24A and 24B), consistent with a role for EphA2 in sustained activation of these pathways. Similarly, atherosclerotic plaques from EphA2 KO mice show reduced phospho-ERK1/2 (FIGS. 7F and 7G) and phospho-Akt1 staining (FIGS. 7H and 7I) in the innominate and carotid arteries (FIGS. 24C-24F) in both the total plaque area and the smooth muscle actin-positive plaque area compared with EphA2 WT controls. Taken together, these results suggest that EphA2 deletion reduces smooth muscle mitogenic signaling, consistent with reductions in smooth muscle and plaque-associated proliferation.

Extracellular matrix deposition and remodeling by intraplaque vascular smooth muscle cells significantly contribute to atherosclerotic plaque progression. Because EphA2 KO mice show reduced progression to advanced-stage plaques at 16 weeks of Western diet feeding (FIG. 5C), the inventors next tested whether EphA2 alters fibrosis, a critical feature of advanced atherosclerosis, in the atherosclerotic plaque. Compared with EphA2 WT mice, EphA2 KO mice exhibited a reduction in plaque collagen content, as evidenced by Masson trichrome (FIGS. 8A and 8B) and polarized Picrosirius red staining (FIGS. 8C and 8D), and a reduction in plaque fibronectin levels (FIGS. 8E and 8F). In addition to reduced fibrous area in the plaque, the percent of plaque area staining positive for Masson trichrome, polarized Picrosirius red, and fibronectin was reduced in EphA2 KO mice (FIGS. 25A-25C). Similar reductions in plaque collagen staining were observed in the aortic root of EphA2 KO mice, suggesting that this reduction in fibrous content extends across multiple vascular beds (FIG. 26). However, analysis of hCoASMC mRNA failed to identify significant differences in matrix gene expression after EphA2 knockdown in vitro (FIG. 27A), although a trend for reduced fibronectin expression was observed on EphA2 depletion. To assess changes in matrix deposition, we isolated the deoxycholate-insoluble matrix from serum-treated smooth muscle cells with or without EphA2 knockdown. Consistent with the in vivo data, EphA2 depletion significantly reduced smooth muscle deposition of deoxycholate-insoluble fibronectin fibrils, including both total fibronectin (FIGS. 8G, 27B, and 27D) and cell-derived fibronectin containing the extradomain A alternative splice site (FIGS. 8H, 27C, and 27E) in both hCoASMCs and in hAoSMCs (FIGS. 28A-28C). Because collagen deposition requires fibronectin polymerization in models of vascular injury, the inventors' data suggest that EphA2 expression may modulate smooth muscle-driven fibrosis in atherosclerotic plaques by regulating fibronectin deposition.

DISCUSSION: Early atherosclerotic plaques are dominated by inflammatory cross-talk between relatively few key cell types, which elicits endothelial cell activation, leukocyte recruitment, and smooth muscle invasion and fibroproliferative remodeling within the developing plaque. The inventors are aware that endothelial EphA2 expression is enhanced and activated during inflammatory contexts, including within the endothelium overlying atherosclerotic plaques. Using the Apoe^{-/-} model of atherogenesis, the inventors now demonstrate that EphA2 deletion reduced atherosclerotic burden associated with reduced inflammation in the early plaque. Although plaque macrophages express EphA2, bone marrow chimeras and in vitro functional studies strongly implicated EphA2 expression in the vessel wall as the proinflammatory and proatherogenic EphA2 pool. ICAM-1 and VCAM-1 expression is attenuated in early atheromas of EphA2 KO mice, consistent with a role for EphA2 in endothelial activation, and blunting endothelial EphA2 expression reduced monocyte firm adhesion in vitro. Whereas EphA2 deletion does not affect the stage of plaque progression at early time points, EphA2 KO mice show significantly reduced late-stage plaque progression associated with reductions in smooth muscle and fibrous tissue content. Consistent with this finding, EphA2 expression is enhanced in activated, synthetic hCoASMCs and hAoSMCs, and blunting EphA2 expression reduces vascular smooth muscle proliferation, mitogenic signaling (ERK1/2, Akt1), and fibronectin deposition both in vivo and in vitro. Together, these data demonstrate that EphA2 expression critically regulates atherosclerotic plaque formation and progression, potentially through dual effects on both inflammation and fibroproliferative remodeling.

Multiple lines of evidence suggest that EphA2 contributes to inflammatory endothelial activation. EphA2 expression is enhanced in a variety of proinflammatory conditions, including atherosclerosis, acute lung injury, ischemia/reperfusion, many cancers, and psoriasis, often concomitant with upregulation of its ligand ephrinA1. Based on the disclosed experiments, reducing or suppressing EphA2 expression is arguably therapeutic for each of these diseases/conditions. Further, the inventors contemplate treatments for these conditions or pre-condition states of these conditions involving administering first therapeutics that reduce or suppress EphA2 expression. The inventors also contemplate a further embodiment where the first therapeutic is combined with a second therapeutic currently known to the art for treating any of the diseases/conditions.

During early atherogenesis, endothelial activation promotes nuclear factor-κB-dependent ICAM-1 and VCAM-1 expression to facilitate leukocyte recruitment and extravasation. EphA2 deletion blunts nuclear factor-κB activation, endothelial permeability, and chemokine expression in lipopolysaccharide-induced lung injury models, and infection of Apoe^{-/-} mice with EphA2 shRNA reduces atherosclerotic lesions and diminishes both nuclear factor-κB activation and proinflammatory gene expression. Atherosclerotic plaques in EphA2 KO mice similarly show reduced VCAM-1 and ICAM-1 expression compared with EphA2 WT mice (FIG. 2D), indicating that EphA2 contributes to the endothelial proinflammatory phenotype in atherosclerosis. Eph/ephrin interactions also regulate leukocyte adhesion/repulsion responses and migration through control of integrin activation and cytoskeletal dynamics. Although EphA2 deletion does not affect TNF-α-induced proinflammatory gene expression, EphA2-depleted endothelial cells show a reduced capacity to support monocyte firm adhesion after TNF-α treatment (FIGS. 4G and 16B). In addition, substrate-bound EphA2 significantly enhances both monocyte adhesion to recombinant ICAM-1/VCAM-1 and monocyte spreading on fibronectin (FIG. 17). Monocyte adhesion and spreading are unaltered when recombinant ephrinA1 is added to the substrates (FIGS. 17A and 17B), further suggesting that monocytic EphA2 does not contribute to monocyte adhesion during leukocyte homing. However, EphA2 does not promote leukocyte recruitment under all contexts because monocyte recruitment during sterile (thioglycollate-elicited) inflammation remains unchanged in EphA2-deficient mice (FIG. 3B). Collectively, these data indicate that endothelial EphA2 expression modulates endothelial-monocyte interactions through multiple mechanisms.

Progression from early fatty streaks to advanced atheromas involves the recruitment of vascular smooth muscle cells into the growing plaque, with fibroproliferative remodeling driving the formation of the protective fibrous cap. To accomplish this, smooth muscle cells undergo a dynamic shift from a quiescent, contractile phenotype in the medial layer to a promigratory, proliferative, and profibrotic phenotype in the atherosclerotic plaque. The inventors disclose herein that smooth muscle transition to this activated phenotype results in the enhanced expression of EphA2 both in cell culture models and in atherosclerotic plaques (FIGS. 5G-5K). Elevated EphA2 expression promotes proliferation and migration in several cancer models, and deletion of EphA2 reduces plaque progression, plaque smooth muscle content, and plaque-associated proliferation, as well as smooth muscle proliferation, mitogenic signaling (phospho-ERK1/2, phospho-Akt1), and scratch wound healing in culture (FIGS. 5-7). Furthermore, deletion of EphA2 reduces plaque fibronectin and collagen content (FIGS. 8A-8F), and EphA2 knockdown in vascular smooth muscle cells attenuates fibronectin deposition (FIGS. 8G and 8H). The reduced collagen content in EphA2-deficient plaques could result from reduced collagen degradation through proteases such as matrix metalloproteinase-2 and -9. Although EphA2 depletion enhances matrix metalloproteinase-2 and -9 mRNA expression, these proteases do not show changes in protein expression or activity after EphA2 knockdown (FIG. 29). Whereas EphA2 can promote fibronectin expression in cancer models, EphA2 did not significantly affect smooth muscle matrix gene expression (FIG. 29), although fibronectin expression was reduced, albeit insignificantly, on EphA2 silencing. Depletion of EphA2 reduces fibronectin deposition (FIGS. 8G, 8H, 27, and 28), and plating smooth muscle cells on a fibronectin matrix abrogates the effect of EphA2 deletion on smooth muscle scratch wound healing (FIG. 30), suggesting that EphA2-dependent fibronectin deposition may contribute to EphA2-dependent regulation of smooth muscle function. Because fibronectin drives vascular smooth muscle proliferation and migration, EphA2-induced fibronectin deposition may promote a permissible environment for vascular smooth muscle infiltration into atherosclerotic lesions. Furthermore, this relationship between EphA2 and fibronectin may incur an unusual feed-forward response because fibronectin alone was shown to upregulate EphA2 expression independently of any other stimulus (FIGS. 5I and 21G). Taken together, these data provide the first report of an EphA2-dependent transition to advanced atherosclerosis through the regulation of smooth muscle fibroproliferative remodeling.

EphA2 appears to play a complex role in the pathogenesis of atherosclerotic cardiovascular disease, with EphA2 deletion inducing opposing effects on plasma lipid levels and plaque formation. Although both male and female EphA2 KO mice display reduced plaque formation (FIGS. 1A-1D), male EphA2 KO mice demonstrated significantly enhanced weight gain and plasma lipid levels when fed either standard chow or a high-fat Western diet (FIGS. 12A-12D). Because EphA2 expression does not affect macrophage lipid uptake and hematopoietic EphA2 deletion does not affect plasma lipid levels in bone marrow chimera (FIGS. 14C 15), the effect of EphA2 deletion on plasma lipid levels may result from a previously unrecognized role for EphA2 in hepatocyte function. Whereas no studies to date have shown a role for EphA2 in metabolic abnormalities, the inventors are aware chromosomal region containing the EphA2 gene (1p36.13) is associated with the metabolic syndrome. Furthermore, genome-wide association analysis links both EphA2 and ephrinA1 to the presence of liver enzymes in plasma, and both EphA2 and ephrinA1 showed reduced expression in a rat model of nonalcoholic fatty liver disease. Although assessment of the role of EphA2 in cholesterol transport responses should serve as an interesting direction for future research, the reduction in atherosclerotic plaque formation despite the elevations in plasma lipid levels underscores the important role of EphA2 signaling in vascular remodeling responses during atherogenesis.

The data shown herein highlight 2 opposing functions of EphA2 in atherosclerosis: the propagation of early atherogenic inflammation and the stabilization of late-stage plaques through fibroproliferative remodeling. However, recent publications highlight the often inverse function of EphA2 signaling resulting from differential ligand-dependent and ligand-independent signaling. Endothelial EphA2 relies on ephrinA1-dependent activation for proinflammatory marker expression and likely interacts with ephrinA1 to promote monocyte firm adhesion (FIGS. 4G and 16). Therefore, ligand-dependent signaling of endothelial EphA2 may contribute to early atherosclerosis by propagating atherogenic inflammation. Although EphA2 ligation inhibits proliferative signaling cascades, enhanced expression of unligated EphA2 promotes migration and proliferation. Unlike endothelial cells, mRNA analysis reveals a downregulation of ephrinA1 in the proliferative vascular smooth muscle phenotype (FIG. 5G), suggesting a potential contribution of ligand-independent signaling from smooth muscle EphA2 in late-stage atherosclerosis. Therefore, the inventors propose pharmacological approaches that limit EphA2 ligand-dependent signaling (including, for example, blocking antibodies, kinase inhibitors) to reduce the proinflammatory effects of EphA2 without inhibiting its beneficial effect on smooth muscle proliferation and extracellular matrix deposition.

EXPANDED MATERIALS AND METHODS: Animals and tissue harvest: Animal protocols were approved by the LSU Health Sciences Center-Shreveport IACUC committee, and all animals were cared for according to the National Institute of Health Guidelines for the Care and Use of Laboratory Animals. EphA2−/− on a mixed C57B1/6J-129S6 background mice were bred onto the C57B1/6J ApoE−/− strain for at least 7 generations. Eight week old, male and female Apoe−/− (EphA2 WT) or EphA2−/− , Apoe−/− (EphA2 KO) mice were fed a high fat, Western-type diet (21% fat by weight; 0.15% cholesterol and 19.5% casein without sodium cholate) for 8, 12, 16, or 24 weeks. Weight and blood glucose were monitored weekly. Glucose was measured with an AlphaTRAK glucometer and AlphaTRAK2 test strips (Abbott Animal Health). After diet, mice were euthanized by pneumothorax under anesthesia and blood was collected by inferior vena cava puncture into heparinized blood collection tubes. Blood was centrifuged at 5000 rpm for 5 minutes, and plasma was isolated and analysis was performed. Total plasma cholesterol (Wako), HDL (Wako), and TG (Pointe Scientific) fractions were measured according to the manufacturer's instructions. LDL was calculated according to Friedewald's equation as LDL=total cholesterol−HDL−(triglycerides÷5). For bone marrow adoptive transfer experiments, 6-8 week old mice were irradiated with 500 rads twice before receiving 1×10⁶ EphA2 WT or EphA2 KO bone marrow cells via retroorbital capillary bed injection. Irradiated mice were maintained on neomycin sulfate water ad libitum for 2 weeks and at 4 weeks post-irradiation were fed the western type diet for 12 additional weeks. Following diet regimens, mice were euthanized, perfused with PBS, and tissues were fixed in 3.7% neutral buffered formaldehyde for analysis of atherosclerotic plaque burden (aorta, aortic root, innominate artery, and carotid arteries). Some aortas were cleaned of adventitia and sonicated in TRIzol for mRNA isolation. For intraplaque angiogenesis studies, 8 week old mice were placed on Western diet for 24 weeks. Five minutes prior to sacrifice, mice were administered biotinylated lectin (100 μg biotinylated lucopersicon esculentum (tomato) lectin in 100 μL 4-(2-hydroxyethyl)-1-piperazineethanesulfonic acid-buffered saline) via retroorbital injection to label perfused vessels. Mice were then sacrificed with isofluorane inhalation and pneumothorax, then perfused with 3.7% neutral buffered formaldehyde. The right carotid artery and aortic root were harvested and embedded in paraffin, then sectioned into 5 μm sections as described.

Cell culture, treatments, and transfections: Human peripheral blood monocytes were isolated according to Institutional Review Board and Health Insurance Portability and Accountability Act guidelines as previously described. Briefly, blood was drawn from healthy volunteers by venipuncture and centrifuged through a Ficol Histopaque 1077 gradient (Sigma). Mononuclear cells were washed with saline, and isolated by centrifugation through a Percoll (Pharmacia) gradient. Cells were washed and re-suspended in serum-free RPMI medium, and used within 24 hours. Peritoneal macrophages were isolated by peritoneal lavage 3-4 days following injection of 1 mL 4% thioglycollate broth and were cultured in RPMI media containing 10% FBS and 100 U/mL penicillin/100 μg/mL streptomycin (Gibco). THP-1 monocytes (ATCC) were cultured in RPMI medium supplemented with 10% denatured fetal bovine serum denatured at 55° C. for 45 minutes, 2 mM glutamine, 100 U/mL penicillin/100 ug/mL streptomycin, and 0.5 mM β-mercaptoethanol. Bone marrow-derived macrophages (BMDM) were grown from bone marrow isolates using DMEM containing 30% L929 cell-conditioned media, 20% FBS, 2 mM glutamine, and 100 U/mL penicillin/100 μg/mL streptomycin. The murine macrophage cell line RAW264.7 (ATCC#TIB-71) was cultured in DMEM medium supplemented with 10% fetal bovine serum (Hyclone), Glutamax (Gibco), 1 mM sodilum pyruvate (Gibco), 100 U/mL penicillin/100 μg/mL streptomycin (Gibco). Human aortic endothelial cells (Lonza) were cultured in EGM-2 growth medium supplemented with the single quots kit, 10% fetal bovine serum (Hyclone) and 100 U/mL penicillin/100 μg/mL streptomycin (Gibco) or MCDB131 medium supplemented with 10% fetal bovine serum (Hyclone), 2 mM glutamine, 176 μg/mL bovine brain extract, 60 μg/mL heparin sodium (Acros), and 100 U/ml penicillin/100 μg/ml streptomycin (Gibco), and used between passages 6-10. Human coronary artery vascular smooth muscle cells (Lonza, Cell Applications) and human aortic vascular smooth muscle cells (Lonza) were cultured in MCDB131 medium supplemented with 10% fetal bovine serum, 5 μg/mL human recombinant insulin (Sigma), 5 ng/mL human recombinant EGF (Peprotech), 5 ng/mL human recombinant FGF-basic (Peprotech), and 100 U/mL penicillin/100 μg/mL streptomycin (Gibco) and used between passages 4-6. As indicated, 6-well plates were coated with fibronectin (10 μg/mL), basement membrane extract (1:50 dilution, Trevigen), collagen I (40 μg/mL), collagen IV (20 μg/mL), or laminin (20 μg/mL) and cells were plated under low serum conditions (serum-free, 0.5% FBS or 1% FBS). HAECs or smooth muscle cells were transiently transfected with Lipofectamine2000 (Invitrogen) according to the manufacturer's instructions and with anti-EphA2 SmartPool siRNA oligonucleotides (Dharmacon) or anti-EphA2 siRNA oligonucleotide (Sigma) for 48 hours prior to experiments. Unless otherwise stated, all experiments with EphA2 siRNA used Dharmacon SmartPool siRNA oligonucleotides. RAW264.7 macrophages (2×10⁶ cells) were transfected with 100 pmoles EphA2 siRNA (Dharmicon SmartPool, Sigma) using the SF Cell Line 4D-Nucleofector™ X Kit (Lonza) according to manufacturer Amaxa™ 4D-Nucleofector™ Protocol for RAW 264.7.

Plaque analysis and immunohistochemistry: Aortas were harvested, cleaned of adventitia, dissected longitudinally along the greater and lesser curvature for bilateral presentation, pinned, and stained with Oil Red O (Alfa Aesar). Vessels were visualized on a DS-Fi1 camera (Nikon) attached to a multizoom AZ100 microscope (Nikon), and plaque burden was analyzed using Nikon Elements software and expressed as a percentage of positive Oil Red O to total aortic area. Immunohistochemistry was performed as previously described. Plaque prone regions, including the aortic sinus, innominate (brachiocephalic) artery, and carotid sinus, were embedded in paraffin and cut in 5 μm sections. Within each staining regimen, sections were taken from the same site equidistant from anatomical landmarks (initiation of the aortic valve leaflets, brachiocephalic branch point, or carotid bifurcation). Plaque analysis involving human tissue was deemed non-human research by the local IRB though exclusive use of post-mortem samples as previously described. Briefly, human atherosclerotic vessels (coronary arteries, left anterior descending artery, left carotid sinus) were harvested during routine autopsy and fixed in 10% formaldehyde. Basic histological stains, including Movat Pentachrome staining, Masson's Trichrome staining, and Picrosirius Red staining was performed using established protocols. Immunohistochemistry was performed with rat anti-Mac2 (1:10,000, Accurate Chemical), goat anti-CD31 (1:50, Santa Cruz), mouse anti-smooth muscle actin (1:400, Sigma), rabbit anti-EphA2 (1:200, Invitrogen), rabbit anti-Ki67 (1:100, Abcam), sheep anti-Von Willebrand factor (1:100, Abcam), rabbit anti-Von Willebrand factor (1:250, Abcam), rabbit anti-phospho-Akt1 (1:100, Cell Signaling Technology), rabbit anti-phospho-ERK1/2 (T202/Y204) (1:200, Cell Signaling Technology), Alexa647-streptavidin (1:1000, Life Technologies), or rabbit anti-phospho-EphA2 (Y772) (1:400, Cell Signaling Technology). Primary antibodies were visualized with either biotinylated secondary antibodies, the Vectastain ABC kit, and 3-3′-diaminobenzidine (DAB, Dako) or with Alexa-Fluor conjugated secondary antibodies (Life technologies) and fluorescence microscopy on a Nikon Eclipse Ti inverted epifluorescence microscope equipped with a Photometrics CoolSNAP120 ES2 camera and NIS Elements 3.00, SP5 imaging software. Quantification of total plaque area or area positive for certain stains were quantified using NIS Elements software.

Quantitative real time PCR: mRNA extraction, cDNA synthesis, and qRT-PCR were performed from cell lysates or tissue as previously described. Briefly, mRNA was extracted with TRIzol reagent and converted to cDNA with the iScript cDNA synthesis kit (Biorad). qRT-PCR was performed in a Biorad iCycler and iQSYBR Green MasterMix (Biorad). Primers (see FIGS. 9 and 10) were designed using online Primer3 software and validated using generic human cDNA pooled from 20 organs (Firstchoice® Total Human RNA, Ambion). PCR products were verified by the presence of a single peak in melt curve analysis and by DNA sequencing (SeqWright). Results were normalized to the housekeeping genes GAPDH, Rpl13a, and/or PPIA as indicated, and expressed as a fold change using the 2-[delta][delta]Ct method.

Lipid Uptake: Peritoneal macrophages isolated from EphA2 WT or EphA2 KO mice were treated with Dil-oxLDL (20 μg/mL, 4 hours). Unbound Dil-oxLDL was removed by washing, and total cellular Dil-oxLDL fluorescence was quantified and normalized to total DAPI. Lipid content in peritoneal macrophages isolated from Western diet-fed (8 weeks) EphA2 WT or EphA2 KO mice was assessed by Nile Red. Total Nile Red fluorescence was quantified and normalized to DAPI. Similarly, Nile Red was utilized to assess lipid content in RAW264.7 macrophages treated with oxLDL (25 μg/mL, 24 hours).

Immunoblotting, immunocytochemistry, and cytokine assays: Cell lysis and immunoblotting was performed as previously described. Membranes were labeled with rabbit anti-EphA2 (1:1000, CST catalogue #6997), rabbit anti-β-tubulin (1:1000, CST), rabbit anti-GAPDH (1:5000, CST), mouse anti-fibronectin-EDA (1:500, Santa Cruz), mouse anti-smooth muscle actin (1:5000), rabbit anti-fibronectin (1:3000, Abcam), rabbit anti-myosin heavy chain (1:1000, Abcam ab53219), rabbit anti-calponin (1:1000, Abcam ab46794), rabbit anti-phospho-Akt1 (S473) (1:1000, Cell Signaling Technology), goat anti-Akt1 (1:500, Santa Cruz), rabbit anti-phospho- ERK1/2 (T202/Y204) (1:1000, Cell Signaling Technology, catalogue #9101), or rabbit anti-ERK1/2 (1:5000, Santa Cruz). Densitometry was performed using ImageJ software. For immunocytochemistry, cells were plated on glass coverslips and treated as described. Cells were fixed with 4% formaldehyde, permeabilized with 0.1% Triton X-100, blocked with 10% goat serum in 1% denatured bovine serum albumin (BSA). and incubated with rabbit anti-Ki67 (1:1000, Abcam) overnight. Cells were incubated with Alexa488-phalloidin (1:400, Invitrogen) to assess cell area and with rabbit anti-Ki67 (1:1000, Abcam) followed by Alexa546-conjugated anti-rabbit IgG secondary antibodies to assess proliferation. For BrdU incorporation, cells were treated as described and treated with BrdU (10 μM, BD Pharmingen) for two hours followed by fixation with 4% formaldehyde. Cells were rinsed twice with 1xPBS and permeabilized with 0.1% Triton X-100. DNA was hydrolyzed with 1M HCl, 2M HCl, and a phosphate/citric acid buffer (182 μM Na₂HPO₄ with 9 μM citric acid, pH 7.4) and incubated with mouse anti-BrdU (1:50, Cell Signaling Technology) for three hours. Cells were washed in TBST and incubated with Alexa647-conjugated goat anti-rabbit or Alexa647-conjugated goat anti-mouse secondary antibody (1:1000) for two hours. Cells were rinsed with TBST to remove unbound secondary antibody and coverslips were mounted onto microslides using Fluoromount G (SouthernBiotech). All stains were visualized on a Nikon Eclipse Ti inverted epifluorescence microscope equipped with a Photometrics CoolSNAP120 ES2 camera and NIS Elements 3.00, SP5 imaging software. At least 100 cells were counted for each experiment and cells were scored for nuclear Ki67 or BrdU and expressed as a percent of total DAPI using NIS Elements software. TNFα in cell culture media was assessed using a commercially-available ELISA assay as previously described.

In vitro monocyte rolling and adhesion assays: To assess monocyte adhesion under static conditions, goat anti-human Fc-coated coverslips were incubated for 2 hours with 1-2 μg/mL Fc-fusion proteins, including Fc-ICAM-1 (0.5 μg/mL), Fc-VCAM-1 (0.5 μg/mL), Fc-ephrinA1 (1 μg/mL) and/or Fc-EphA2 (1 μg/mL). In some experiments, fibronectin (1 μg/mL) was also utilized. Coverslips were washed in PBS and incubated with 0.2% BSA for 30 minutes prior to use. THP-1 monocytes were labeled with Cell Tracker Green CMFDA (Invitrogen) for 15 minutes, washed, and plated onto the recombinant proteins for either 10 minutes to assess adhesion or 1 hour to assess cell spreading. Analysis of human peripheral blood monocyte cell rolling and arrest was performed as previously described. Briefly, HAECs were transfected with EphA2 siRNA and treated with TNFα (10 ng/mL, Sigma) for four hours and assembled onto a with a GlycoTech parallel plate flow chamber (Rockville, Md.). Human peripheral blood monocytes or THP-1 monocytes were labeled with Cell Tracker Green CMFDA (Invitrogen) for 15 minutes, washed by centrifugation, and re-suspended to 2-2.5×10⁵ cells/mL in HBSS containing calcium and magnesium. Monocytes were stirred in a glass beaker at 60 rpm and 37° C., and perfused over endothelial cell monolayers at physiological flow rate of 1.5 dynes/cm² with a digital syringe pump. Rolling velocity of fluorescently labeled cells was calculated by motion tracking of single cells using Simple PCI software (Compix). Images were captured with a Nikon Eclipse TE-2000 epifluorescent microscope and Hamamatsu digital camera at 29 frames/second. Firmly adherent cells were defined as a cell that did not move more than one cell diameter within a five second period, as determined by software analysis and manual review.

Scratch Wound Assay: Human coronary artery vascular smooth muscle cells were serum starved for three days to obtain synchronization, then maintained in serum-free conditions or 1% serum conditions and treated with mock or siEphA2. 24 hours post-transfection, cells were plated at approximately 42,000cells/cm² in a 12-well plate coated with either fibronectin (10 μg/mL) or collagen I (40 μg/mL). A scratch was created with a P200 pipette tip, and cells were maintained (37° C.; 5% CO₂) in a stage mounted environment chamber from Bioscience Tools (San Diego, Calif.). Wounds were imaged in a 1.5×1.5 mm field (3 fields per wound) at 30-minute intervals for 18 hours using a motorized stage attached to a Nikon TiE microscope controlled by NIS elements advanced research software package using a Nikon monochrome cooled digital camera (DSQi1). Wound closure was analyzed with NIS-Elements 4.0 software. Wound closure was calculated as percent closure from t=0.

Fibronectin fibrillogenesis: Analysis of fibronectin deposition and fibrillogenesis was performed as previously described. For Western blot analysis, cells were lysed in a 2% deoxycholate buffer (2% sodium deoxycholate, 20 mM tris base, 2 mM PMSF, 2 mM iodoacetic acid, 2 mM Nethylmaleimide, pH 8.8) and deoxycholate-insoluble material was isolated by centrifugation (16,000×g) for 15 minutes. The supernatant was kept as the deoxycholate-insoluble fraction. Deoxycholate-insoluble material includes deposited and/or assembled extracellular matrix, while the deoxycholate-soluble fraction represents intracellular material. Both fractions were lysed in 2× Laemmli buffer and analyzed by Western blotting as previously described. For immunocytochemical analysis, cells were rinsed with PBS and extracted by sequential washes of 3% Triton X-100 in PBS and 2% deoxycholate extraction buffer (2% sodium deoxycholate, 50 mM tris base, 10 mM EDTA, pH 8.9) and rinsed with PBS prior to fixation with 4% formaldehyde. Cells were blocked with 10% goat serum in 1% BSA, then incubated with mouse anti-fibronectin-EDA (1:500, Santa Cruz) or rabbit anti-fibronectin (1:500, Santa Cruz) overnight. Cells were washed in TBST and incubated with Alexa647-conjugated goat anti-rabbit or Alexa547-conjugated goat antimouse secondary antibody (1:1000) for two hours. Cells were rinsed with TBST to remove unbound secondary antibody and coverslips were mounted onto microslides using Fluoromount G (SouthernBiotech). Stains were visualized on a Nikon Eclipse Ti inverted epifluorescence microscope equipped with a Photometrics CoolSNAP120 ES2 camera and NIS Elements 3.00, SP5 imaging software. Fibronectin fibrils were quantified as fold area.

Gelatin Zymography: MMP activity was assessed with a commercially available gelatin zymography assay (BioRad). Cells were lysed in Zymogram Sample Buffer (BioRad #1610764) and passed through a 25 gauge needle 5 times to shear DNA. Approximately 8 μg of protein was loaded into each well of a zymogram gel (BioRad #1611167) and lysates were separated with gel electrophoresis. Zymogram gels were renatured in Zymogram Renaturation Buffer (BioRad #1610765) for 1 hour, then developed at 37° C. overnight in Zymogram Development Buffer (BioRad #1610766). Following overnight incubation, gels were stained with Coomassie blue (BioRad #1610436) for 30 minutes, then destained with 30% ethanol/10% acetic acid until clear bands became visible. Gels were imaged using a Biorad ChemiDoc Touch Imaging System. Densitometry was performed using ImageJ software.

Statistical Analyses: All results shown are means±standard error (SEM) with the number of experiments performed provided in the figure legends. Statistical analyses were performed using GraphPad Prism software. Statistical significance was determined by Student's T tests, one-way ANOVA with Bonferroni post-tests, two-way ANOVA with Bonferroni post-tests, or Chi squared tests. Data was considered significantly different when the p-value was less than 0.05.

Animal Models and Tissue Harvest: Animal protocols were approved by the LSU Health Sciences Center Animal Care and Use Committee, and all animals were cared for according to the National Institute of Health's Guidelines for the Care and Use of Laboratory Animals. Apoe^{-/-} mice were crossed with EphA2^{+/+}or EphA2^{-/-} mice and fed Western diet for 8, 12, 16, or 24 weeks. Weight and blood glucose were monitored weekly. After the diet regimens, mice were euthanized and perfused with PBS, and tissues were fixed in 3.7% neutral-buffered formaldehyde before paraffin embedding or staining with Oil Red O. Some vessels were used for mRNA extraction before fixation.

### Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary). In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017).

The methods described herein can include the administration of a therapeutic, or prodrugs or pharmaceutical compositions thereof, or other therapeutic agents. Exemplary therapeutics include those to suppress EphA2 expression (including gene therapy and anti-miRNA treatments) and inhibition of ephrin type-A receptor 2.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

### Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

### Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

### Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

### Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

### Dosing Regimes

The present methods for treating proinflammatory or fibroproliferative conditions are carried out by administering a therapeutic for a time and in an amount sufficient to result in decreased inflammation or decreased fibrosis or decreased EphA2 expression.

The amount and frequency of administration of the compositions can vary depending on, for example, what is being administered, the state of the patient, and the manner of administration. In therapeutic applications, compositions can be administered to a patient suffering from proinflammatory or fibroproliferative condition in an amount sufficient to relieve or least partially relieve the symptoms of the proinflammatory or fibroproliferative condition and its complications. The dosage is likely to depend on such variables as the type and extent of progression of the proinflammatory or fibroproliferative condition, the severity of the proinflammatory or fibroproliferative condition, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the proinflammatory or fibroproliferative condition or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the proinflammatory or fibroproliferative condition.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating a condition in a mammal comprising:
administering a pharmacologically effective amount of a therapeutic;
wherein the therapeutic one of decreases EphA2 expression and inhibits ephrin type-A receptor 2; and
the condition is a proinflammatory or fibroproliferative condition.

2. The method of claim 1 wherein the proinflammatory or fibroproliferative conditions is one of atherosclerosis, acute lung injury, ischemia/reperfusion, cancer and psoriasis.

3. The method of claim 1 wherein the condition is atherosclerosis.

4. The method of claim 1 wherein the therapeutic further with inhibits ligand ephrinA1.

5. The method of claim 1 wherein the therapeutic limits EphA2 ligand-dependent signaling.

6. The method of claim 1 wherein the therapeutic is one of a blocking antibody and a kinase inhibitor.

7. The method of claim 1 wherein the therapeutic decreases EphA2 expression.

8. The method of claim 7 wherein the therapeutic decreases EphA2 expression through gene therapy.

9. The method of claim 7 wherein the therapeutic increases EphA2 expression through anti-miRNA treatments.

11. The method of claim 1 wherein the mammal is a human.

12. A pharmaceutical composition for treating proinflammatory or fibroproliferative condition comprising;
a first therapeutic that one of decreases EphA2 expression, inhibits ligand ephrinA1, and both decreases; and EphA2 expression, inhibits ligand ephrinA
a second distinct therapeutic.

13. The pharmaceutical composition of claim 11 wherein the second distinct therapeutic is one of one that treats plasma cholesterol level and treats elevated plasma triglyceride level.

14. The pharmaceutical composition of claim 11 wherein the second distinct therapeutic is a statin.

15. The pharmaceutical composition of claim 11 wherein the second distinct therapeutic is a thereapeutic for treating a proinflammatory or fibroproliferative condition.

16. The pharmaceutical composition of claim 11 wherein the proinflammatory or fibroproliferative condition is one of atherosclerosis, acute lung injury, ischemia/reperfusion, cancer, and psoriasis.


SEQUENCE LISTING (104 sequences)

SEQ ID NO: 1 - DNA, 19 bp, Mus musculus
gaaggtgaaggtcggagtc

SEQ ID NO: 2 - DNA, 20 bp, Mus musculus
gaagatggtgatgggatttc

SEQ ID NO: 3 - DNA, 20 bp, Mus musculus
atgacaagaaaaagcggatg

SEQ ID NO: 4 - DNA, 20 bp, Mus musculus
cttttctgcctgtttccgta

SEQ ID NO: 5 - DNA, 20 bp, Mus musculus
atgaggggaccacatctacg

SEQ ID NO: 6 - DNA, 20 bp, Mus musculus
cacctggattcctttttcca

SEQ ID NO: 7 - DNA, 20 bp, Mus musculus
tgtccccctcaaaagtcatc

SEQ ID NO: 8 - DNA, 20 bp, Mus musculus
taggcaacggggtctctatg

SEQ ID NO: 9 - DNA, 21 bp, Mus musculus
tgtctgatcttgctaggaccg

SEQ ID NO: 10 - DNA, 22 bp, Mus musculus
gagagtaacggcctttttgtga

SEQ ID NO: 11 - DNA, 21 bp, Mus musculus
atgcggtggaacactttctgg

SEQ ID NO: 12 - DNA, 21 bp, Mus musculus
gcacgtcaactctacactggt

SEQ ID NO: 13 - DNA, 21 bp, Mus musculus
tgtccacatctcgttctcggt

SEQ ID NO: 14 - DNA, 22 bp, Mus musculus
ccgctctcgttggtatttctga

SEQ ID NO: 15 - DNA, 23 bp, Mus musculus
atgatggcttattacagtggcaa

SEQ ID NO: 16 - DNA, 20 bp, Mus musculus
gtcggagattcgtagctgga

SEQ ID NO: 17 - DNA, 22 bp, Mus musculus
cctctctctaatcagccctctg

SEQ ID NO: 18 - DNA, 21 bp, Mus musculus
gaggacctgggagtagatgag

SEQ ID NO: 19 - DNA, 21 bp, Mus musculus
ctgacccagaagcgtcacttg

SEQ ID NO: 20 - DNA, 22 bp, Mus musculus
acagattcatagacagtcccca

SEQ ID NO: 21 - DNA, 21 bp, Mus musculus
catggaagcggtctctgtgat

SEQ ID NO: 22 - DNA, 23 bp, Mus musculus
gctttgatagcagttggacatcc

SEQ ID NO: 23 - DNA, 23 bp, Mus musculus
tcttttctcccagccccaggaca

SEQ ID NO: 24 - DNA, 23 bp, Mus musculus
acagccagtgacagccatcccag

SEQ ID NO: 25 - DNA, 23 bp, Mus musculus
gactttaagggttacctgggttg

SEQ ID NO: 26 - DNA, 21 bp, Mus musculus
tcacatgcgccttgatgtctg

SEQ ID NO: 27 - DNA, 21 bp, Mus musculus
gtgtggcgctagtgtctgtaa

SEQ ID NO: 28 - DNA, 21 bp, Mus musculus
cacgttccagctacttcaacc

SEQ ID NO: 29 - DNA, 20 bp, Mus musculus
tagttagagggtaggttttg

SEQ ID NO: 30 - DNA, 19 bp, Mus musculus
ctcattctttccacaagta

SEQ ID NO: 31 - DNA, 22 bp, Mus musculus
ttctccatctccggtgaaaaca

SEQ ID NO: 32 - DNA, 21 bp, Mus musculus
acctcccgaccagaacatagg

SEQ ID NO: 33 - DNA, 20 bp, Mus musculus
tggattttgcgacgctgtca

SEQ ID NO: 34 - DNA, 20 bp, Mus musculus
cacttcctcccaccctcctt

SEQ ID NO: 35 - DNA, 21 bp, Mus musculus
aaggaaccctgtggctattgg

SEQ ID NO: 36 - DNA, 23 bp, Mus musculus
gcaaacatgcccgttttagagaa

SEQ ID NO: 37 - DNA, 21 bp, Mus musculus
tgcgaagtccggcaatttctt

SEQ ID NO: 38 - DNA, 22 bp, Mus musculus
gcaacacaacttggttggagac

SEQ ID NO: 39 - DNA, 20 bp, Mus musculus
ccggggaagagagatgttgc

SEQ ID NO: 40 - DNA, 21 bp, Mus musculus
caaattgccccatgatgcttg

SEQ ID NO: 41 - DNA, 21 bp, Mus musculus
ggcgaagtgaacttgttggat

SEQ ID NO: 42 - DNA, 21 bp, Mus musculus
aaggactcgtctacctcgttg

SEQ ID NO: 43 - DNA, 21 bp, Mus musculus
cgccgaagaagttatcctcct

SEQ ID NO: 44 - DNA, 19 bp, Mus musculus
gggacggtcgtgttcatcc

SEQ ID NO: 45 - DNA, 22 bp, Mus musculus
cttcacgccttttatcttgggc

SEQ ID NO: 46 - DNA, 23 bp, Mus musculus
tggggattatgagtgattttgcc

SEQ ID NO: 47 - DNA, 21 bp, Mus musculus
cgatacgcagtctactggaac

SEQ ID NO: 48 - DNA, 21 bp, Mus musculus
ggtagtcgttgatgctcacct

SEQ ID NO: 49 - DNA, 19 bp, Mus musculus
cgtgcaggtgaacgtgaac

SEQ ID NO: 50 - DNA, 21 bp, Mus musculus
gtaacgctggaacttctcgga

SEQ ID NO: 51 - DNA, 19 bp, Mus musculus
agagggggcaaatgccttc

SEQ ID NO: 52 - DNA, 21 bp, Mus musculus
ctgagaatcgaacagggctga

SEQ ID NO: 53 - DNA, 21 bp, Mus musculus
acacgtccaaagggttcaaga

SEQ ID NO: 54 - DNA, 21 bp, Mus musculus
cggtgtcatctgctgttctaa

SEQ ID NO: 55 - DNA, 19 bp, Homo sapiens
gaaggtgaaggtcggagtc

SEQ ID NO: 56 - DNA, 20 bp, Homo sapiens
gaagatggtgatgggatttc

SEQ ID NO: 57 - DNA, 20 bp, Homo sapiens
gcctgacaccacatacatcg

SEQ ID NO: 58 - DNA, 20 bp, Homo sapiens
gaaaacgagaatcccaagca

SEQ ID NO: 59 - DNA, 20 bp, Homo sapiens
atgaccaacgacgacatcaa

SEQ ID NO: 60 - DNA, 20 bp, Homo sapiens
gcagggggaggaaagaacta

SEQ ID NO: 61 - DNA, 20 bp, Homo sapiens
ccagttcccgtgtaaagcac

SEQ ID NO: 62 - DNA, 20 bp, Homo sapiens
cccacccatccaataaatga

SEQ ID NO: 63 - DNA, 20 bp, Homo sapiens
tttcgccctattttcgtgtc

SEQ ID NO: 64 - DNA, 20 bp, Homo sapiens
gttattctggctgggttcca

SEQ ID NO: 65 - DNA, 20 bp, Homo sapiens
ctacaccacaaggggaggaa

SEQ ID NO: 66 - DNA, 20 bp, Homo sapiens
agagcagcaggacaatccat

SEQ ID NO: 67 - DNA, 20 bp, Homo sapiens
atgccccttcacgtgtcgta

SEQ ID NO: 68 - DNA, 20 bp, Homo sapiens
aaccagccaatctccatcag

SEQ ID NO: 69 - DNA, 20 bp, Homo sapiens
cagattcgggcttttactgc

SEQ ID NO: 70 - DNA, 20 bp, Homo sapiens
agtgccttctcccaatgatg

SEQ ID NO: 71 - DNA, 20 bp, Homo sapiens
cagcacacaagaaagccagt

SEQ ID NO: 72 - DNA, 20 bp, Homo sapiens
agatgcggagagagaggatg

SEQ ID NO: 73 - DNA, 21 bp, Homo sapiens
ttgaaaaggaccaagagacca

SEQ ID NO: 74 - DNA, 20 bp, Homo sapiens
aaccactgccaacaaaatga

SEQ ID NO: 75 - DNA, 20 bp, Homo sapiens
aggctgaagagagggacagg

SEQ ID NO: 76 - DNA, 20 bp, Homo sapiens
aggaatggtgggaaaggact

SEQ ID NO: 77 - DNA, 18 bp, Homo sapiens
acggtggaggtgagcatc

SEQ ID NO: 78 - DNA, 18 bp, Homo sapiens
agggagaagggcgtgaag

SEQ ID NO: 79 - DNA, 20 bp, Homo sapiens
ctctgggctacgagttccac

SEQ ID NO: 80 - DNA, 20 bp, Homo sapiens
ccgctgatgctcttctcaag

SEQ ID NO: 81 - DNA, 20 bp, Homo sapiens
ctccaggtgtctgtctgctg

SEQ ID NO: 82 - DNA, 20 bp, Homo sapiens
cttgggatgagagggaaggt

SEQ ID NO: 83 - DNA, 21 bp, Homo sapiens
ccagaagataagactgagcgc

SEQ ID NO: 84 - DNA, 22 bp, Homo sapiens
ccattatctgggattgcagagg

SEQ ID NO: 85 - DNA, 17 bp, Homo sapiens
tgcctggtgaacgtggt

SEQ ID NO: 86 - DNA, 19 bp, Homo sapiens
aggagagccatcagcacct

SEQ ID NO: 87 - DNA, 24 bp, Homo sapiens
ggcaaacatggaaaccgtggtgaa

SEQ ID NO: 88 - DNA, 24 bp, Homo sapiens
ggcagaccttgcaatccattgtgt

SEQ ID NO: 89 - DNA, 24 bp, Homo sapiens
tatcgaacacgcaaggctgtgaga

SEQ ID NO: 90 - DNA, 24 bp, Homo sapiens
ggccaacgtccacaccaaattctt

SEQ ID NO: 91 - DNA, 21 bp, Homo sapiens
cagtggacacacttagccctc

SEQ ID NO: 92 - DNA, 20 bp, Homo sapiens
gcggcattctctggaactcc

SEQ ID NO: 93 - DNA, 20 bp, Homo sapiens
tcgaggaggaaattccaatg

SEQ ID NO: 94 - DNA, 20 bp, Homo sapiens
ctcttcatgacgcttgtgga

SEQ ID NO: 95 - DNA, 19 bp, Homo sapiens
gcgtgtagcacctgaagag

SEQ ID NO: 96 - DNA, 20 bp, Homo sapiens
gaatggcgacgtacatggca

SEQ ID NO: 97 - DNA, 19 bp, Homo sapiens
cgccaagagactcgtctgg

SEQ ID NO: 98 - DNA, 21 bp, Homo sapiens
tctttcccaaccgtgaccttc

SEQ ID NO: 99 - DNA, 21 bp, Homo sapiens
ctgtcagccgaggttaagaac

SEQ ID NO: 100 - DNA, 21 bp, Homo sapiens
gaggccgtccatgaagttgtt

SEQ ID NO: 101 - DNA, 20 bp, Homo sapiens
aacggacaaagagttggcag

SEQ ID NO: 102 - DNA, 20 bp, Homo sapiens
gagcaaaggcatcatccact

SEQ ID NO: 103 - DNA, 22 bp, Homo sapiens
tgtaccgctatggttacactcg

SEQ ID NO: 104 - DNA, 19 bp, Homo sapiens
ggcagggacagttgcttct